(19) United States
(12) Patent Application Publication
(10) Pub. No.: US 2025/0344015 A1
(43) Pub. Date: Nov. 6, 2025

(54) USER SPECIFIC ACTIVE SOUND-REDUCTION, HEARING PROTECTION SYSTEM, AND RELATED COMPUTER PRODUCTS AND METHODS

(71) Applicant: Sound United, LLC, Carlsbad, CA (US)

(72) Inventors: Jose Andres Lobo, Owings Mills, MD (US); Jonathan Rodman, Parkville, MD (US)

(21) Appl. No.: 19/197,844

(22) Filed: May 2, 2025

Related U.S. Application Data
(60) Provisional application No. 63/642,296, filed on May 3, 2024.

(51) Int. Cl. H04R 1/10; G10K 11/178

(52) U.S. Cl. CPC H04R 1/1083; G10K 11/17825; G10K 11/17881; H04R 1/1016; G10K 2210/1081

(57) ABSTRACT

An active hearing protection system can generate audio signals adapted in real time for a specific user is provided with internal (e.g., within the ear canal) and external microphones, along with methods of making and using the hearing protection system. The System and method can be configured to Continually monitor Total SPL by internal transducer or microphone; and if user-specific safe SPL level is exceeded, the system can reduce a long-term threshold for the subject user. The method includes recording said subject user's Total SPL exposure data; and in response to said user's Total SPL exposure data, automatically determining updated user-specific or personalized optimal sound characteristics for said user to cater to the user's then-extant unique hearing.

### CROSS-REFERENCE TO RELATED APPLICATIONS

This non-provisional patent application claims priority under 35 U.S.C. 119 (e) to U.S. Provisional Patent Application No. 63/642,296, filed on May 3, 2024, and entitled “USER SPECIFIC ACTIVE SOUND-REDUCTION, HEARING PROTECTION SYSTEM, AND RELATED COMPUTER PRODUCTS AND METHODS,” which is hereby incorporated by reference herein in its entirety.

### INCORPORATION BY REFERENCE

Portions of this application are generally related to U.S. Pat. No. 9,794,672, which issued on Oct. 17, 2017, U.S. Pat. No. 10,708,680, which issued on Jul. 7, 2020, and U.S. Pat. No. 10,154,333, which issued on Dec. 11, 2018, which are each incorporated herein by reference in their entirety.

### BACKGROUND

Embodiments disclosed herein relate to user-wearable hearing protection systems, especially automatic hearing protection systems, such as those including devices worn in or on one or both of a wearer's ears.

Audio headphones are generally designed to transduce an electronic input signal to an acoustic wave across a frequency range. An example of audio headphones is disclosed in U.S. Pat. No. 10,708,680, FIG. 3.

In an embodiment of U.S. Pat. No. 10,708,680, otoacoustic emissions (“OAEs”) of a user are measured and playback is adjusted based on the measurement of OAEs via a highly sensitive microphone integrated into in-ear headphones.

### OBJECTS AND SUMMARY

This Summary is provided to introduce a selection of representative concepts in a simplified form, which representative concepts are further described below in the Detailed Description of Example Embodiments. This Summary is not intended to identify key features or essential features of the pseudo-claim statements, nor is it intended to be used to limit the scope of the claims.

Accordingly, it is an object of certain example embodiments to overcome one or more (or all) of the above-mentioned difficulties by providing a device, system, and method that provide automatic control over the SPL a user is exposed to over time, such as while maintaining safe levels (e.g., audiologist-recognized safe levels) of sound over time. It is another object to maintain such safe levels while achieving high frequency reproduction and low frequency attenuation to substantially preserve the fidelity of signals.

In a first aspect, an active sound-reduction, hearing protection system is provided. The hearing protection system includes, at least, a first earpiece wearable in an ear canal of a subject, memory, and a digital signal processing (DSP) module operatively coupled with the earpiece and the memory. The earpiece can be configured as an In-Ear Monitor (“IEM”) that seals into the user's ear canal with there's a first microphone on the outside (exposed to the ambient environment) and sealed within the ear canal a driver (as often used in conventional IEMs or earbuds) and mounted in very close proximity to that driver there's a second microphone positioned within the ear canal to sensing sound leakage from the outside as well as playback sound from the driver. In an example embodiment, the IEM or earpiece housing also encloses and carries a Digital Signal Processing (“DSP”) module (such as on a semiconductor chip in the housing).

The earpiece includes a passive attenuation structure (e.g., a sealing eartip) configured to sit at least partially within the ear canal when the earpiece is worn, a first input transducer (e.g., an external microphone) configured to be acoustically coupled to environmental surroundings of the user or subject when the earpiece is worn to receive environmental sounds and generate a first input audio signal corresponding to the environmental sounds, an output transducer (e.g., a driver) configured to be acoustically coupled to an inner volume of the user's ear canal when the earpiece is worn to receive an output audio signal and generate playback output sounds based on the output audio signal that are emitted into the ear canal, and a second input transducer (e.g., an internal microphone) configured to be acoustically coupled to the inner volume of the ear canal when the earpiece is worn to receive an acoustic sum comprising the playback output sounds and leakage sounds and generate a second input audio signal, wherein the leakage sounds originate from the environmental surroundings yet are not acoustically blocked from the inner volume of the ear canal by the earpiece. The DSP module is configured to apply short-term sound reduction to the first input audio signal and/or the second input audio signal in response to transient high-level sound pressure levels, accumulate an exposure history of the ear canal (using the second input audio signal) to the playback output sounds and the leakage sounds over a period of time, record the exposure history in the memory, apply long-term sound reduction to the first input audio signal and/or the second input audio signal in response to non-transient high-level sound pressure levels based upon the user's actual exposure history, and generate the output audio signal based on the application of the short-term sound reduction and the long-term sound reduction.

A second aspect provides a digital signal processing (DSP) module including a computer-readable storage medium and program code embodied on the computer-readable storage medium. The program code is executable by a processor to effectively provide a user-specific or personalized personal volume knob with perfect fidelity in an active hearing protection device that automatically controls SPL the user is exposed to over time.

A third aspect provides a computer-implemented method, comprising providing an active sound-reduction, hearing protection system. The system can be programmed to perform a sequence of operations, when in use, including the following method steps:
(a) Turn on device;
(b) Sense a leakage level using the internal transducer or microphone;
(c) Determine and Set a high pass Fc and long-term compressor threshold or output gain attenuation for the user;
(d) Feed an external transducer signal through, for example, a 2 stage compressor (full or multiband);
(e) Play back the Compressed signal through the driver transducer into the user's ear canal;
(f) Determine or Calculate: Driver Output+Ear Plug Leakage=Total SPL (where total SPL in the user's ear canal is acoustically Summed at the internal transducer microphone and also at the user's ear drum);
(h) Continually monitor Total SPL by internal transducer or microphone; and
(i) If user-specific safe level is exceeded, Reduce long term threshold for subject user, where, in the case of multiband compression, for example the internal mic signal does not feed the long-term compressor threshold (of step (c)), but instead feeds the output gain attenuation for total SPL.

The user-personalized or user specific active sound-reduction hearing protection system includes, at least, a first earpiece wearable in an ear canal of a subject, memory, and a digital signal processing (DSP) module operatively coupled with the earpiece and the memory. The earpiece includes a passive attenuation structure (e.g., an earbud) configured to sit at least partially within the ear canal when the earpiece is worn, a first input transducer (e.g., an external microphone) configured to be acoustically coupled to environmental surroundings of the subject when the earpiece is worn to receive environmental sounds and generate a first input audio signal corresponding to the environmental sounds, an output transducer (e.g., a driver) configured to be acoustically coupled to an inner volume of the ear canal when the earpiece is worn to receive an output audio signal and generate playback output sounds based on the output audio signal that are emitted into the ear canal when the earpiece is worn, and a second input transducer (e.g., an internal microphone) configured to be acoustically coupled to the inner volume of the ear canal when the earpiece is worn to receive an acoustic sum comprising the playback output sounds and leakage sounds and generate a second input audio signal, wherein the leakage sounds originate from the environmental surroundings yet are not acoustically blocked from the inner volume of the ear canal by the earpiece. The DSP module is configured to receive the second input audio signal (from within the ear canal's enclosed volume) and determine an accumulated SPL exposure history for the user, where the exposure includes both environmental leakage within the ear canal and whatever sound is generated by the earpiece's internal driver/speaker, such that where all of the sound within the ear canal is sensed to determine accumulated exposure, continuously. The DSP module is further configured to apply long-term sound reduction to the first input audio signal and/or the second input audio signal in response to non-transient high-level sound pressure levels based upon the exposure history, and generate the output audio signal based on the application of, at least, the long-term sound reduction to the first input audio signal and/or the second input audio signal.

A fourth aspect provides a hearing protection system including a wearable earpiece and a digital signal processing (DSP) module. The wearable earpiece comprises a passive attenuation structure (e.g., an earbud) configured to sit at least partially within the ear canal when the earpiece is worn, a first input transducer (e.g., an external microphone) configured to be acoustically coupled to environmental surroundings of the subject when the earpiece is worn to receive environmental sounds, an output transducer (e.g., a driver) configured to be acoustically coupled to an inner volume of the ear canal when the earpiece is worn, and a second input transducer (e.g., an internal microphone) configured to be acoustically coupled to the inner volume of the ear canal when the earpiece is worn. The DSP module is operatively connected to the first and second input transducers and the output transducer to monitor exposure of the wearer to sound pressure levels, use the output transducer to reproduce high frequencies of the detected sound pressure level inside the ear canal of the wearer with feed forward/feedback gain and compression signal processing, determine passive attenuation of the earpiece, and/or attenuate low frequencies of the sound pressure level that leak through the passive attenuation structure using a feedforward/feedback partial destructive interference.

In a fifth aspect, a hearing protection system comprises a wearable earpiece that includes, at least, a passive attenuation structure (e.g., an earbud) configured to sit at least partially within the ear canal when the earpiece is worn, a first input transducer (e.g., an external microphone) configured to be acoustically coupled to environmental surroundings of the subject when the earpiece is worn to receive environmental sounds, an output transducer (e.g., a driver) configured to be acoustically coupled to an inner volume of the ear canal when the earpiece is worn, and a second input transducer (e.g., an internal microphone) configured to be acoustically coupled to the inner volume of the ear canal when the earpiece is worn. A digital signal processor (DSP) module is configured to process high frequencies lost to passive attenuation and reproduce the high frequencies using the output transducer. The DSP module is configured to attenuate low frequencies that leak through the passive attenuation structure via partial destructive interference by introducing captured low frequency signals captured by the first and/or second first input transducer. In an embodiment, the low frequency signals are captured primarily via the at least one first input transducer, although in another embodiment the low frequency signals are captured primarily via the at least one second input transducer, or in another embodiment via both the first and second input transducers. According to an example embodiment of the aspect, based on the amount of sound pressure levels and time the user is exposed to, the hearing protection system is configured to automatically reduce sound pressure at the ear canal to a desired level.

A sixth aspect provides a hearing protection system comprising a wearable earpiece including, at least, a passive attenuation structure (e.g., an earbud) configured to sit at least partially within the ear canal when the earpiece is worn, a first input transducer (e.g., an external microphone) configured to be acoustically coupled to environmental surroundings of the subject when the earpiece is worn to receive environmental sounds, an output transducer (e.g., a driver) configured to be acoustically coupled to an inner volume of the ear canal when the earpiece is worn, and a second input transducer (e.g., an internal microphone) configured to be acoustically coupled to the inner volume of the ear canal when the earpiece is worn. The hearing protection system further includes a digital signal processing (DSP) module configured to process high frequencies lost to passive attenuation and reproduce the high frequencies using the output transducer. The DSP module is further configured to attenuate low frequencies that leak through the passive attenuation via partial destructive interference. In an embodiment, the low frequency signals are captured primarily via the at least one first input transducer, although in another embodiment the low frequency signals are captured primarily via the at least one second input transducer, or in another embodiment via both the first and second input transducers. According to an example embodiment, DSP module is configured to utilize active sound pressure level monitoring and a control algorithm. Based on Sound Pressure Levels (SPLs) and an amount of time the wearer is subject to the SPLs, the hearing protection system is configured to automatically reduce sound pressure at the ear canal to a desired level.

According to one or more embodiments optionally encompassed by the above aspects, high frequency sounds lost to passive attenuation are processed and reproduced for playing by the audio output transducer into the ear of the listener.

According to one or more embodiments optionally encompassed by the above aspects, low frequency sounds that leak through the passive attenuation structure are attenuated by the DSP module by practicing, for example, partial destructive interference. In a related embodiment, partial destructive interference is applied only when SPL levels are so loud that passive attenuation requires further “assistance” to protect the user's hearing.

According to one or more embodiments optionally encompassed by the above aspects, the hearing protection system is configured to automatically reduce SPLs at the ear canal to a desired and/or safe level. The system optionally includes first and second or left side and right side earpieces adapted to be worn in a user's left ear canal and right ear canal, respectively.

According to one or more embodiments optionally encompassed by the above aspects, SPL-dosage logging in conjunction with an “in-ear” device is provided that utilizes an active sound reduction algorithm.

According to one or more embodiments optionally encompassed by the above aspects, a device is provided that uses periodic audiogram logging that analyzes hearing over time.

According to one or more embodiments optionally encompassed by the above aspects, a device is provided using sound pressure level metering with a custom weighting calculation generated with the listener's otoacoustically derived audiogram (e.g., a custom weighting tool or calculation for metering and processing the signal fed into the individual user's ear canal, responding in real time).

According to one or more embodiments optionally encompassed by the above aspects, the input transducers and/or the output transducer comprise an internal digital-to-analog converter or an internal analog-to-digital converter.

According to one or more embodiments optionally encompassed by the above aspects, the input transducers and/or the output transducer comprise an external (e.g., operatively associated) digital-to-analog converter or an external (e.g., operatively associated) analogy-to-digital converter.

Some embodiments disclosed herein can relate to a hearing protection system, which can include an earpiece wearable in an ear canal of a subject, the earpiece comprising: a passive attenuation structure; an external microphone positioned to receive environmental sounds when the earpiece is worn and configured to generate a first input audio signal corresponding to the environmental sounds; an output transducer positioned and configured to deliver playback output sounds into the ear based on an output audio signal when the earpiece is worn; and an internal microphone positioned to receive an acoustic sum comprising the playback output sounds and leakage sounds that include environmental sounds not acoustically blocked from the ear canal by passive attenuation structure of the earpiece, and configured to generate a second input audio signal corresponding to the acoustic sum. The system can include memory and a digital signal processing (DSP) module operatively coupled to the memory.

The DSP module can be configured to use the second input audio signal to accumulate an exposure history of the ear to the acoustic sum over a period of time, and record the exposure history in the memory, apply long-term sound reduction to the first input audio signal and/or the second input audio signal in response to sound pressure levels based at least in part on the exposure history, and generate the output audio signal based on the application of at least the long-term sound reduction to the first input audio signal and/or the second input audio signal.

The DSP module can be configured to apply short-term sound reduction to the first input audio signal and/or the second input audio signal in response to transient sound pressure levels (e.g., above a threshold). The short-term sound reduction can include short-term compression, and the long-term sound reduction can include long-term compression and/or attenuation. The short-term sound reduction can include single-band short-term compression. The long-term sound reduction can include single-band long-term compression. The short-term sound reduction can include short-term multi-band compression. The long-term sound reduction can include long-term multi-band attenuation. The long-term sound reduction can include attenuation via partial destructive interference of frequencies of the second input audio signal corresponding to the leakage sounds (e.g., the frequencies can be low frequencies, such as below a threshold). The DSP module can be configured to, using the second input audio signal, reproduce frequencies of the environmental sounds that are lost to passive attenuation (e.g., the frequencies can be high frequencies, such as above a threshold). The period of time can be within a range of 1 hour to 24 hours. The period of time can be greater than 24 hours. The external microphone and the internal microphone can share a common analog-to-digital converter. The DSP module can be configured to sense a leakage level using at least the internal microphone. The DSP module can be configured to apply more compression to a lower range of frequencies and to apply less or no compression to a higher range of frequencies. The DSP module can be configured to determine a direction of a sound and to determine an amount of sound reduction to the sound based at least in part on the determined direction of the sound. The hearing protection system can be configured to switch between an active noise cancelation mode and an active sound reduction mode.

Some embodiment disclosed herein can relate to a hearing protection method, which can include providing a first earpiece wearable in a first ear of a subject. The first earpiece can include a passive attenuation structure; an external microphone positioned to receive environmental sounds when the earpiece is worn and configured to generate a first input audio signal corresponding to the environmental sounds; an output transducer positioned and configured to deliver playback output sounds into the ear based on an output audio signal when the earpiece is worn; and an internal microphone positioned to receive an acoustic sum comprising the playback output sounds and leakage sounds that include environmental sounds not acoustically blocked from the ear canal by passive attenuation structure of the earpiece, and configured to generate a second input audio signal corresponding to the acoustic sum. The method can include using a digital signal processing (DSP) module and memory to: use the second input audio signal to accumulate an exposure history of the ear to the acoustic sum over a period of time, and record the exposure history in the memory, apply long-term sound reduction to the first input audio signal and/or the second input audio signal in response to sound pressure levels based at least in part on the exposure history, and generate the output audio signal based on the application of at least the long-term sound reduction to the first input audio signal and/or the second input audio signal. The hearing protection method can include using the DSP module to apply short-term sound reduction to the first input audio signal and/or the second input audio signal in response to transient sound pressure levels (e.g., above a threshold).

It should be understood that the above-described aspects and embodiments may be combined with one another in any combination and may be modified to include, for example, one or more embodiments described herein, including in the detailed description below and in the accompanying drawings. It should also be understood that the above-described aspects and embodiments may be practiced in connection with methods of making and using the device.

The above and still further objects, features and advantages of certain aspects and embodiments will become apparent upon consideration of the following detailed description of example embodiments, particularly when taken in conjunction with the accompanying drawings, wherein like reference numerals in the various figures are utilized to designate like components.

### BRIEF DESCRIPTION OF THE SEVERAL VIEWS OF THE DRAWINGS

The drawings referenced herein form a part of the specification and are incorporated herein by reference. Features shown in the drawings are meant as illustrative of one or more embodiments, and not necessarily of all embodiments, unless otherwise explicitly indicated.

FIG. 1 illustrates a known noise cancelling music playback earbud arrangement in operative relation to a wearer's ear, showing the earbud at partially least partially inserted into the ear canal of the wearer.

FIG. 2A illustrates an early prototype of applicants' Multi-Function Active Hearing Protection (“MFAH”) system and their test fixture with a diagram depicting an earpiece in the user's ear as used when developing an example embodiment.

FIG. 2B depicts a passive attenuation structure (e.g., a flanged silicon ear tip) of the earpiece of FIG. 2A and FIGS. 2C-2F are diagrams illustrating the configuration for components within and on the earpiece of FIG. 2A, in accordance with an example embodiment.

FIG. 3 illustrates a flow diagram of signal flow including a digital signal processing (DSP) module according to an example embodiment.

FIG. 4 illustrates a flow diagram of an example embodiment of active sound reduction (ASR) compression suitable for the DSP module of FIG. 3, in which the ASR compression involves full-band compression.

FIG. 5 illustrates a flow diagram of another example embodiment of active sound reduction (ASR) compression suitable for the DSP module of FIG. 3, in which the ASR compression involves multi-band compression.

FIG. 6 illustrates a flow diagram of an example embodiment of ASR low frequency (LF) attenuation suitable for the DSP module of FIG. 3.

FIG. 7 illustrates a flow diagram of an example embodiment of directionality processing suitable for the DSP module of FIG. 3.

FIG. 8 illustrates a flow diagram of an example embodiment of device voicing suitable for the DSP module of FIG. 3.

FIG. 9 illustrates a flow diagram of an example embodiment of user EQ suitable for the DSP module of FIG. 3.

FIG. 10 illustrates a flow diagram of an example embodiment of corrective hearing for the DSP module of FIG. 3.

FIG. 11 illustrates simulated spectrum analysis plots for (a) a typical environmental or ambient sound field (‘as is”) and (b) the sound resulting from operation of the system where the signal is conditioned with ASR Low Frequency Attenuation.

FIG. 12 is a graph illustrating sound reduction for a passive attenuation, foam-style hearing protection system exposed to 115 dB SPL, wherein the horizontal axis represents frequency (Hz, starting at 20 Hz) and the left-side vertical axis represents SPL in decibels (dB).

FIG. 13 is a graph illustrating sound reduction for a high frequency reproduction hearing protection system exposed to 115 dB SPL, wherein the horizontal axis represents frequency (Hz, starting at 20 Hz) and the left-side vertical axis represents SPL in decibels (dB).

FIG. 14 is a graph illustrating Active Sound Reduction (ASR) for an in-ear hearing protection system according to an example embodiment exposed to 115 dB SPL, wherein the horizontal axis represents frequency (Hz) and the left-side vertical axis represents SPL in decibels (dB).

FIG. 15 is a graph illustrating ASR for an in-ear hearing protection system according to an example embodiment exposed to environmental SPL at 90 dB, wherein the horizontal axis represents frequency (Hz) and the left-side vertical axis represents SPL in decibels (dB).

FIG. 16 is a graph illustrating ASR for a hearing protection system according to an example embodiment exposed to environmental SPL at 100 dB SPL, wherein the horizontal axis represents frequency (Hz, starting at 20 Hz) and the left-side vertical axis represents SPL in decibels (dB).

FIG. 17 is a graph illustrating ASR for an in-ear hearing protection system according to an example embodiment exposed to environmental SPL at 120 dB SPL, wherein the horizontal axis represents frequency (Hz) and the left-side vertical axis represents SPL in decibels (dB).

FIG. 18 is a digital simulation of frequency response inside a simulated ear canal with an active sound reduction (ASR) system processing a stimulus, wherein the horizontal axis represents frequency (Hz) and the left-side vertical axis represents decibels (dB).

FIG. 19 illustrates an embodiment of partial destructive interference of a sine wave when needed because SPL levels are so loud that more than passive attenuation is desired to protect the wearer/user.

FIG. 20 is a diagram showing a hearing protection device worn on an ear of a user according to an example embodiment.

FIG. 21 is a diagram showing a hearing protection device worn on an ear of a user according to another example embodiment.

FIG. 22 is a pictorial display of a hearing protection system according to an example embodiment, and in particular an in-ear device with foam tips (passive attenuation) and high frequency reproduction (partial ASR) resulting in hi fidelity sound reduction in a moderate range of SPL, in communication with a testing station.

FIG. 23 is a schematic diagram representing a system including signal flow according to an example embodiment.

FIG. 24 is a diagram illustrating an example embodiment of the signal processing Decision Tree for use in the Multi-Function Active Hearing Protection (“MFAH”) method.

### DETAILED DESCRIPTION OF EXAMPLE EMBODIMENTS

It will be readily understood that the components and features of the example embodiments, as generally described herein and illustrated in the Figures, may be arranged and designed in a wide variety of different configurations. Thus, the following detailed description of the embodiments of the methods, devices, assemblies, apparatus, systems, modules, submodules, etc. of the example embodiments, as presented in the Figures, is not intended to limit the scope of the embodiments, as recited in the accompanying claims, but is merely representative of selected embodiments.

The illustrated embodiments will be best understood by reference to the drawings, wherein like parts are generally designated by like numerals throughout. The following description is intended only by way of example, and illustrates certain selected embodiments of methods, devices, assemblies, apparatus, systems, etc.

Reference throughout this specification to “a select embodiment,” “one embodiment,” “an example embodiment,” “example embodiments,” “an embodiment,” or “embodiments” means that a particular feature, structure, or characteristic described in connection with the embodiment(s) is included in at least one embodiment. Thus, appearances of the phrases “in a select embodiment,” “in one embodiment,” “in an example embodiment,” “in example embodiments,” “in an embodiment,” or “in embodiments” in various places throughout this specification are not necessarily referring to the same embodiment(s) or only a single embodiment. The embodiments may be, for example, combined with one another in various combinations and modified to include features of one another.

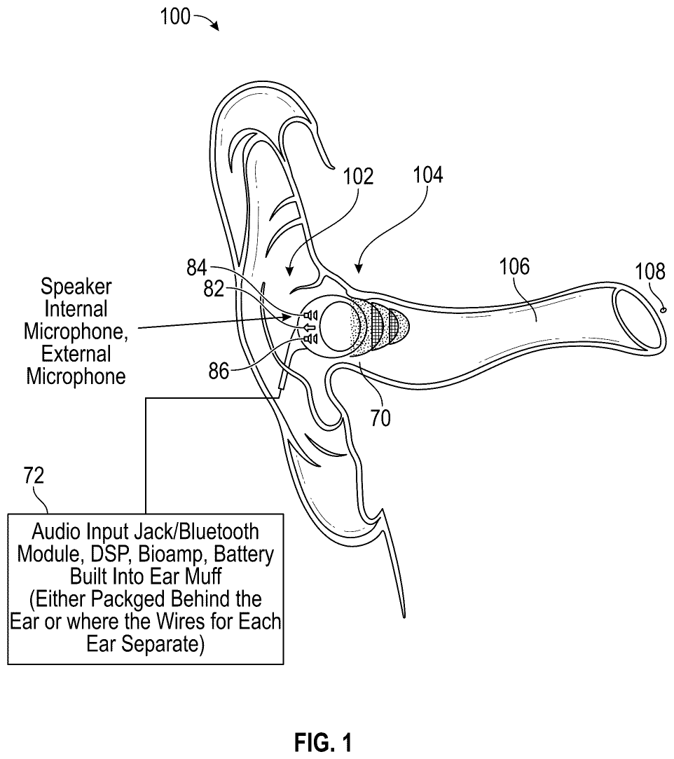

FIG. 1 of the accompanying drawings illustrates an earbud 70 arrangement adapted to be located within the ear canal of one of a user's ears. The anatomy of the ear 100 includes the concha 102 which defines the fleshy portion at the entry of the external ear canal 104, which is in fluid communication with the internal portion of the lumen of the ear canal 106 which terminates within the scull at the ear drum 108. The earbud arrangement 70 includes two speakers 84 and 86, an internal microphone 82, and an optional external microphone. The earbud 70 is connected to an electronics module 72. In an embodiment, the earbud 70 uses an OAE measurement to adjust the playback of the earbud 70 by operation of a digital sound processor (DSP) so that playback performance (e.g., of recorded music) is adapted to the individual (OAE adjusted) needs of the user is provided. The earbud 70 can transmit pilot sound into the ear and capture the cochlea response using the internal microphone. Then, a hearing profile is automatically generated, and acoustic sound and music are adapted to the personalized profile.

A problem not addressed by some “noise cancelling” earphones is that modern lifestyles expose people to unsafe environmental sound pressure levels (SPLs) over a prolonged period. Indeed, an increase in urbanization throughout the world has created high SPL environments. High SPL environments increase the risk of immediate hearing loss to those exposed to such high SPL environments.

The World Health Organization's (WHO's) recent World Report on Hearing indicates that 1.5 billion people worldwide have some degree of hearing loss, and that 430 million people need audiological healthcare. Out of that group, less than 3% (on average worldwide) use hearing aids. The percentage of that group that has used any type of hearing protection for any amount of time is unknown.

Negative health effects other than hearing loss caused by high SPL environments include the risk of ischemic heart disease, hypertension, tinnitus, and cognitive impairments. Applicant has developed products and services that assist in the protection and conservation of hearing. For example, the Masimo Rainbow SET™ platform can assist in preventing hypoxia in neonates by ensuring proper oxygenation is maintained. The Radical-7™ Pulse Co-oximeter can assist in preventing hyperbilirubinemia through the assessment of spectrophotometric hemoglobin in neonates. If left undetected, these events can permanently damage the auditory system of neonates.

A sound pressure level reduction device may directly improve the lives of wearers of such a device. Conventional passive and active hearing protection devices act to reduce exposure of high sound pressure levels to lessen damage to the hearing ability of wearers of the devices. However, such conventional devices are characterized by a loss of fidelity to high frequency roll-off and low frequency leakage.

It would be an advantage to provide a solution to conserve the fidelity of the signals listened to, such as a concert performance, while maintaining the listener at a safe level of listening and reducing the exposure of high sound pressure levels (SPLs), especially those SPLs that can damage their hearing ability, such as to audiologist-recognized safe levels of dosage over time.

It would also be an advantage to develop a holistic monitoring ecosystem that strengthens and empowers the individual listener to conserve their audiology system from birth to elder adulthood by providing a product focused on hearing protection of the ear. Reducing the exposure of high sound pressure level not only contributes to the conservation of one's hearing, but also mitigates other negative health effects from this exposure. Such negative health effects include the risk of ischemic heart disease, hypertension, tinnitus, and cognitive impairments. For those who suffer from hyperacusis or autistic overstimulation, a sound pressure level reduction device may directly improve their lives.

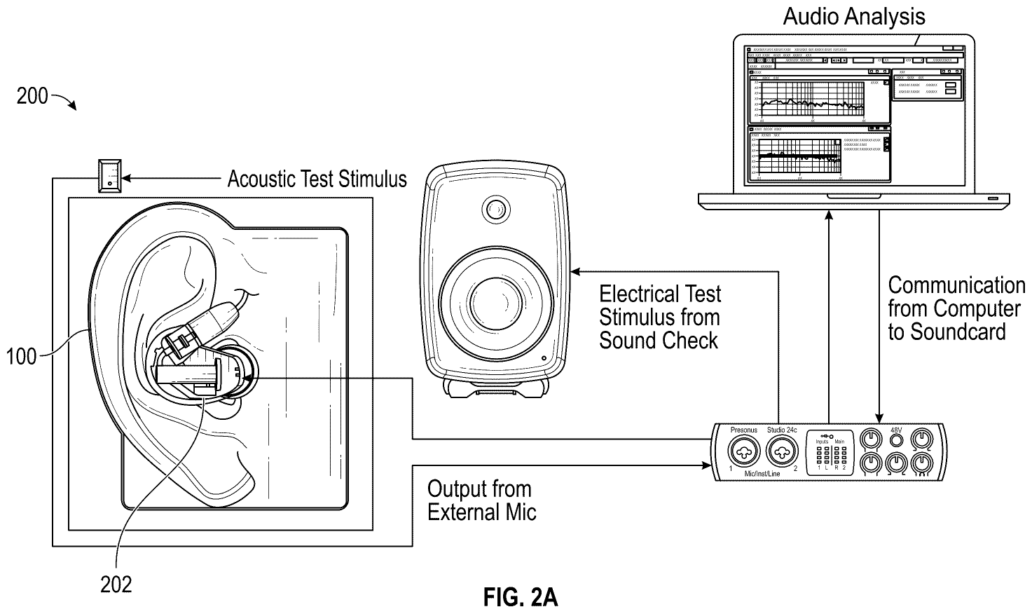

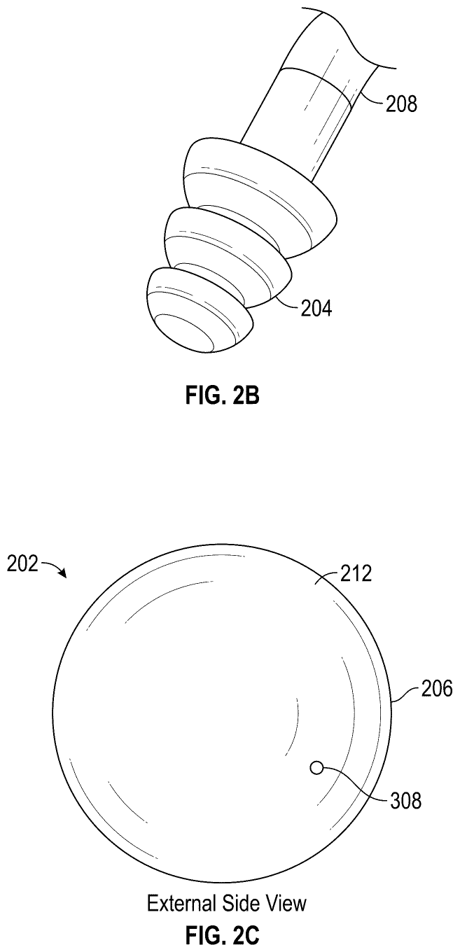

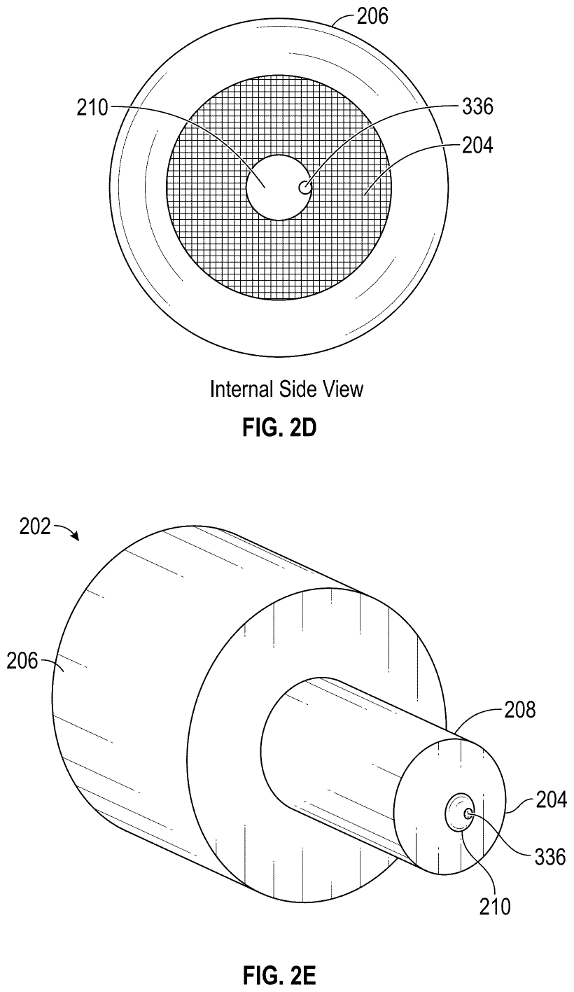

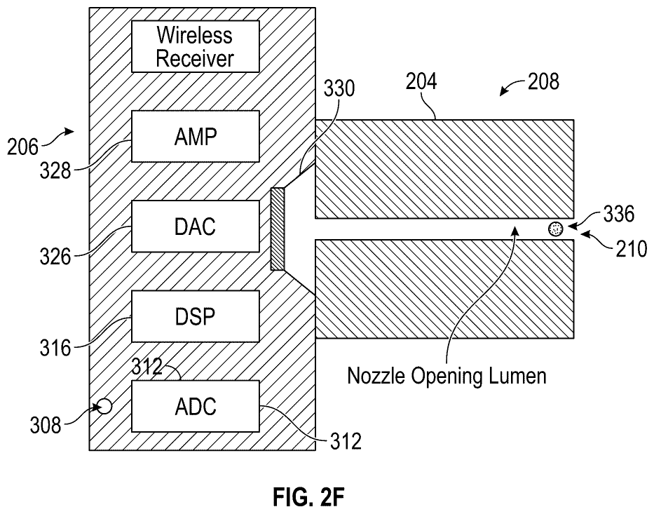

FIG. 2A illustrates an embodiment of a hearing protection system generally designated by reference numeral 200. The hearing protection system 200 includes an earpiece (or earphone) 202, which can be embodied as an earbud that is sized and shaped to fit at least partially, or completely, into the external ear canal of a wearer.

As illustrated in FIG. 2A, the earpiece 202 can include a passive attenuation structure 204, which is shown in FIG. 2B and discussed in further detail herein. The earpiece 202 can be configured as an In-Ear Monitor (“IEM”) that seals into the user's ear canal, such as with a first microphone on the outside (e.g., exposed to the ambient environment). Sealed within the ear canal can be a driver. In some implementations, mounted in very close proximity to that IEM driver can be a second internal microphone configured to be positioned and, when in use, sealed within the ear canal to sense (a) sound leakage from the outside as well as (b) playback sound from the driver. In an example embodiment, the IEM or earpiece housing also encloses and carries a Digital Signal Processing (“DSP”) module (e.g., on a chip within the earbud housing).

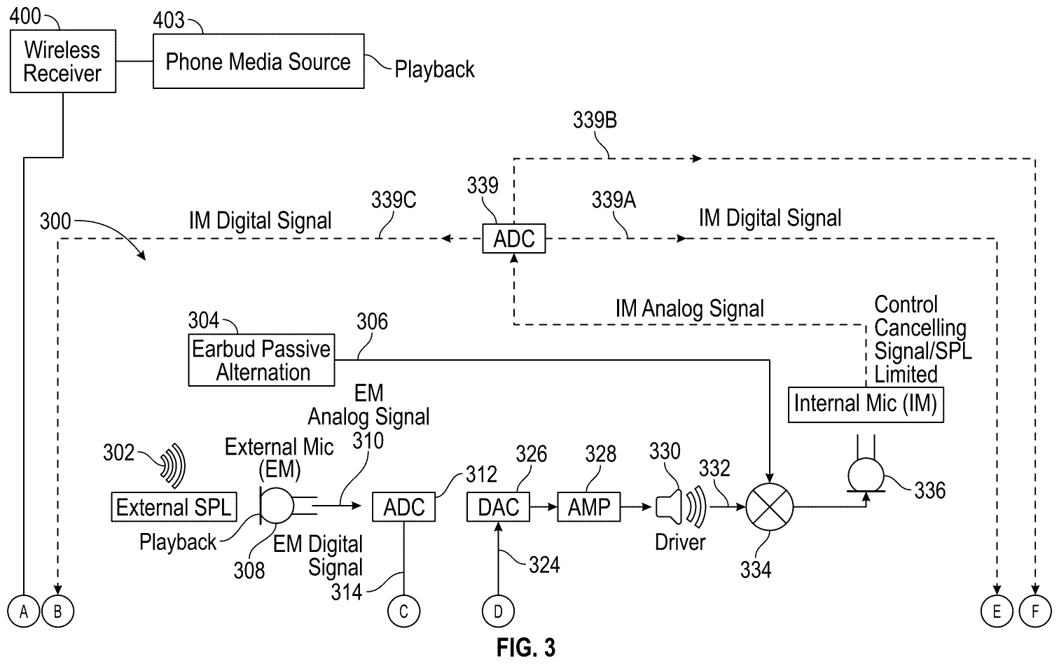

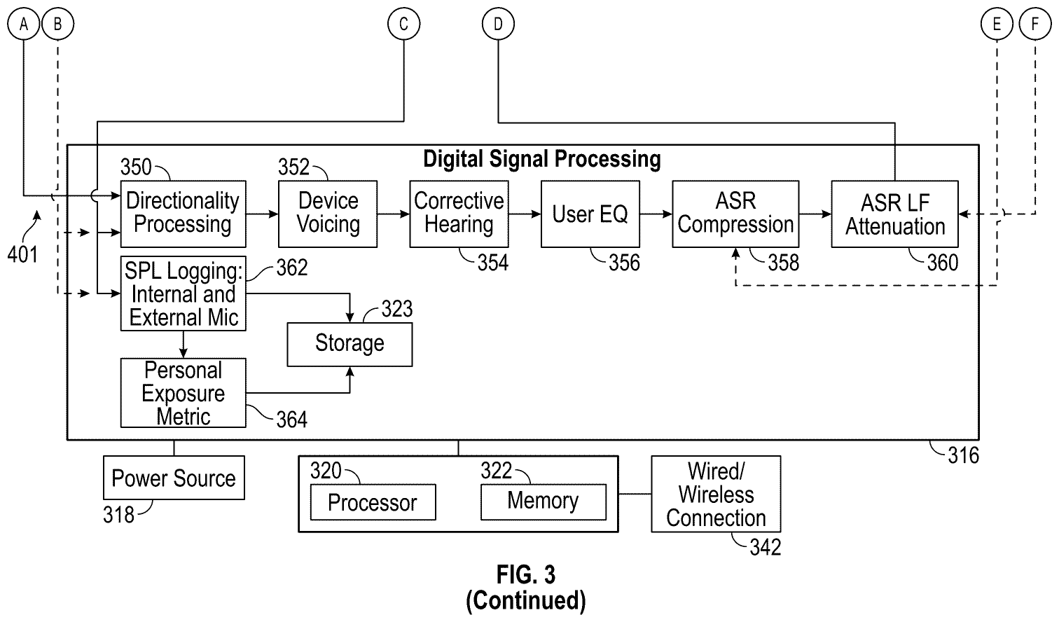

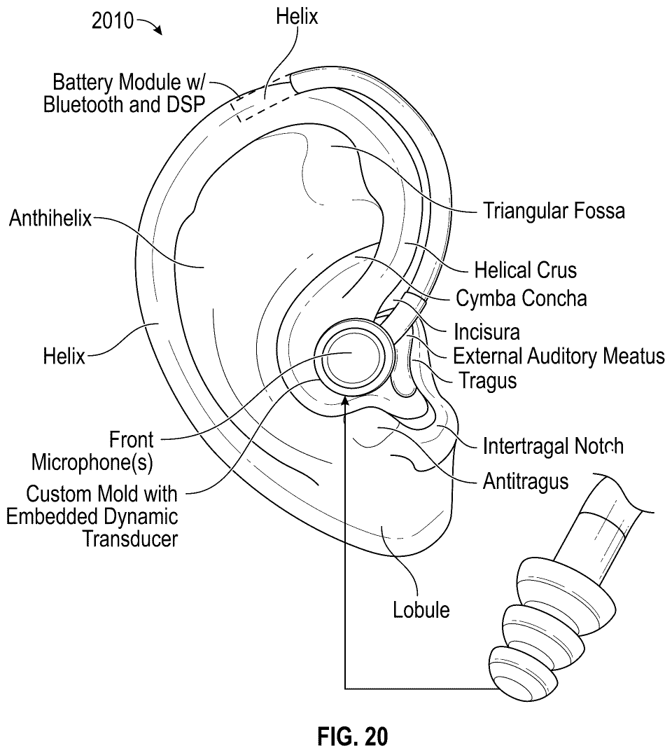

Turning now to the illustrations of FIGS. 20, 2D, 2E and 2F, FIG. 2C shows housing 206 from the external or proximal side, while FIG. 2D illustrates housing 206 and nozzle 210 from the distal or internal side, with a view into the lumen opening 210 showing the position of internal microphone 336. Earpiece or earbud 202, when in use, sits in and is surrounded by the user's concha 102, for example, and provides a proximal or outward facing housing surface 212 from which is aimed an external microphone 308. Housing 206 also carries an inwardly or distally projecting tubular nozzle structure 208 which projects into the user's external ear canal 106 and provides support for a sealing passive attenuation structure, for example made of a foam plug or ear tip 204, when in use. The distally/inwardly projecting tubular nozzle structure 208 can define a fluid impermeable sidewall surrounding nozzle lumen 210 for fluid communication from a dynamic driver 330 (see, e.g., FIG. 3) within housing 206 that projects sound into the ear canal 106. Earpiece nozzle 208 also carries, aims and/or supports an internal microphone 336, such as positioned at the inner or distal edge of the tubular structure at lumen distal opening 210, so that internal microphone 336 can sense the actual acoustic energy present in the wearer's external ear canal 106 which comprises both ambient sound that has leaked or traveled past the passive attenuation or ear tip structure 204 and the sound produced by the dynamic driver 330. Earbud housing 206 can also include a wireless receiver 400, an amplifier module 328, a digital to analog conversion module 326, and/or a digital signal processing module 316 along with an analog to digital conversion module 312, which receives inputs from, among other things, at least one external microphone (e.g., 308) positioned to be exposed to ambient noise 302 from the proximal or exterior surface of earbud housing 206.

FIG. 2B illustrates an example of the ear canal sealing passive attenuation structure 204 of the earpiece 202 of FIG. 2A, and in the illustrated embodiment comprises a flanged rubber, elastomer or silicon ear tip structure having one or more circumferential flanges surrounding a central lumen defining sidewall that is open at both ends. Passive attenuation structure 204 could also be configured as a sealing foam ear tip member with compressible foam surrounding a central lumen defining sidewall that is open at both ends and sized to be compressed to fit easily within a user's ear canal 106 to then expand, engage and seal the user's ear canal 106 from the environment outside the ear, thereby passively attenuating sound 302 from the surrounding environment. Any suitable structure can be used to attenuate the transmission of external sounds into the ear canal.

The hearing protection systems (e.g., 200 and 300, discussed herein) are described in some embodiments herein as being adapted for wearing in a single ear 100 of the user (where the other ear is blocked or protected from damaging sounds). It should be understood that implementation of various methods described herein may involve equipping each of the right and left ears of the wearer with a respective (left and right) individual hearing protection system components. The left and right hearing protection systems may operate independently or dependently with one another. For example, the left and right hearing protection systems may independently monitor and record ambient external sound pressure levels (SPLs) and in-ear canal SPLs. As another example, the left and right hearing protection systems may independently or dependently make changes, modifications, or corrections, including for addressing high level SPLs. According to another example, the left and right hearing protection systems may have separate DSP modules or share a common DSP module.

FIG. 3 is a component level schematic diagram illustrating signal flow in an active sound-reduction, hearing protection system generally designated by reference numeral 300 which may be configured as is system 200 in FIGS. 2A-2F. The hearing protection system 300 is shown in relation to a user's ambient environment including environmental sounds 302. Environmental sounds 302 may include, for example and without limitation, music, speech, audio and audio-video media, ambient sounds, industrial noises, animal sounds, recreational or military occupational sounds (e.g., gunshots) and other sounds experienced by a user in daily life. Referring generally to FIGS. 3-23, hearing protection system 300 includes a passive attenuation structure 304 carried on an earpiece (e.g., an earbud), similar to that illustrated in FIG. 2A. Earpiece 304 can be configured as an In-Ear Monitor (“IEM”) that seals into the user's ear canal with a first microphone 308 on the outside (exposed to the ambient environment 302) and sealed within the ear canal is a driver 330. Mounted in very close proximity to driver 330 is a second internal microphone 336 positioned within the ear canal to sense sound leakage from the outside as well as playback sound from driver 330. In an example embodiment, the IEM or earpiece housing also encloses and carries Digital Signal Processing (“DSP”) module 316 (such as on a preprogrammed chip in the earbud housing).

The ear canal sealing passive attenuation structure 304 may be, for example, an earbud foam tip, eartip, or an earplug (in the same manner as passive attenuation structure 204). Representative passive attenuation structures 304 include, without limitation, ergonomically sealable foams, silicones, and silicone blends, with or without flanges. The signal flow for an example earbud carrying a passive attenuation structure 304 is illustrated in FIG. 3. For the purposes of FIGS. 3-10, the acoustic effects of passive attenuation structure 304 are considered as part of an earbud or IEM housing (e.g., 206) situated in a wearer's concha 102, although it should be understood that the acoustic effects of earbud/passive attenuator structure (designated as 304 in FIG. 3) is not thereby limited. The IEM housing (e.g. 206) included with passive attenuation structure 304 can be configured to sit within the concha 102 and at least partially, and in some cases completely, within the external ear canal 104 of a wearer, such as a user or other listener, when the earpiece is worn inserted to sealably engage the interior surfaces of and project sound into the user's ear canal 106.

Known passive attenuation devices, including earbuds/earplugs, often do not make a perfect seal with the ear canal of the user/listener when worn. As a result, some portion of the ambient or environmental sounds 302 are not blocked by the passive attenuation structure 304 and consequently leak past the passive attenuation structure 304 into ear canal 106 as leakage sounds 306 (see, e.g., FIG. 3). That is, the leakage sounds 306 originate from the environmental surroundings (i.e., the environmental sounds 302) yet are not acoustically blocked from the inner volume of the ear canal 106 by the earbud/passive attenuation structure 304. The leakage sounds 306 and their treatment by various example embodiments are described in greater detail below.

The hearing protection system 300 further includes a first input transducer 308, which is illustrated in FIGS. 3-10 as comprising at least one external acoustic sensor or microphone 308. Although only a single external microphone 308 is depicted in FIG. 3, it may be desirable to provide the hearing protection system 300 with more than one external microphone. For example, a two-ear system would include at least a first or left external microphone 308 for a first or left earpiece (or earbud) and a second or right external microphone for a second or right earpiece (or earbud). Alternatively, each earpiece (or earbud) could carry multiple (e.g., frontwardly and rearwardly aimed) directional external microphones and directionality processing circuitry 350 to provide improved directionality operations and optionally for noise cancellation operations.

The first input transducer (e.g., external microphone 308) is configured to acoustically couple to environmental surroundings of the listener when the earpiece is worn. Thus, the first input transducer or external microphone 308 receives the environmental sounds 302 outside of an inner volume of the ear canal 106, e.g., on an outer side of the passive attenuation structure 304 opposite the ear canal, when in use. The first input transducer 308 is further configured to generate a first (external microphone) input audio signal 310 corresponding to the environmental sounds 302 received or sensed by the first input transducer. In FIG. 3, the first input audio signal 310 is an analog signal, for example. As discussed below, the first input transducer (external mic. 308) is operatively associated with an analog-to-digital converter (ADC) 312, which converts the first input transducer's signal 310 into a digital signal 314. Alternatively, it should be understood that the first input transducer 308 may include an internal ADC 312 for producing the first input audio (external microphone) signal 310 as a digital signal 314, which can be transmitted as a digital input signal to digital signal processing (DSP) module 316.

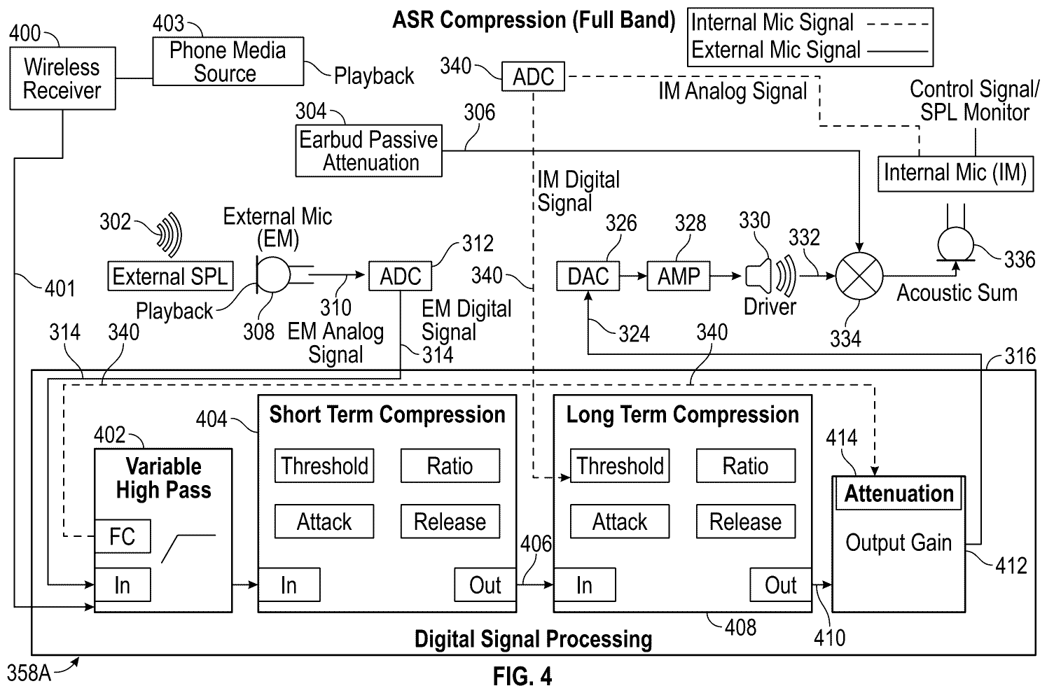

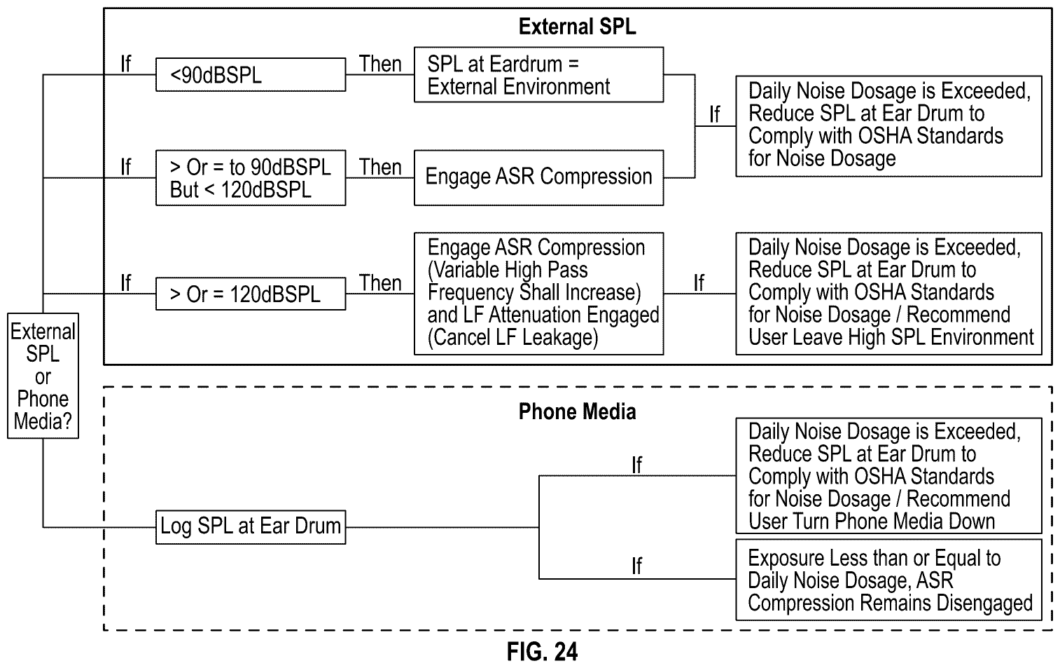

The DSP module 316 is shown in FIG. 3 as operatively connected to a power source 318 (such as a battery) for providing power to the hearing protection system 300, including the DSP module 316. FIG. 3 also illustrates the DSP module operatively connected to a processor 320 and a memory 322. (For the sake of convenience, the power source 318, the processor 320, and the memory 322 are only depicted in FIG. 3 and are omitted from FIGS. 4-10.) The memory 322 may store, for example and without limitation, programming instructions and data used by the processor 320 during program execution, including short-term compression parameters and values, long-term compression parameters and values, attenuation parameters and values, device voicing parameters and values, User EQ parameters and values, corrective hearing parameters and values, logged long-term SPL information, programming related to the decision tree method illustrated in FIG. 24, and other information.

In FIG. 3, the memory 322 can be local, such as local storage 323. In another embodiment, the processor 320 and/or the memory 322 can be a remote processing unit and/or database (e.g., loaded on a smartphone 403 or a computer or a tablet) connected to the DSP module 316 using wired or wireless connections, such as connection 342 or wireless receiver 400, described below.

The processor 320 and memory 322 are associated with the wired and/or wireless connection 342. The wired/wireless connection 342 can be used to enable communication to and from the hearing protection system 300. For example, the wired/wireless connection 342 can be used to receive digital and/or analog input signals, transfer data to and from the DSP module 316 (e.g., to an external resource or physician, such as an audiologist), and/or change stored setting, parameters, and/or values stored in the memory 322.

Further features of the DSP module 316 are described below, including in connection with FIGS. 4-10. For now, it suffices to point out that the DSP module 316 outputs a digital output audio signal 324.

The digital output audio signal 324 is transmitted to a digital-to-analog converter (DAC) 326, which delivers the resulting analog output audio signal to an amplifier 328. The amplified analog output audio signal is received by an output transducer 330, which in the Figures is depicted as an electrodynamic driver, but may be a speaker or other transducer for converting the amplified electrical audio signals from amplifier 328 into playback output sounds 332. As in the case of ADC 312 discussed above, the DAC may be separate from or integrated into the output transducer 330. That is, the output transducer 330 may be configured to generate the digital output signal by integrating the output transducer 330 with the DAC 326 or by being operatively associated with the DAC 326.

The output transducer 330 is configured to be acoustically coupled to and project sound directly into the inner volume of the ear canal 106 when the earpiece (e.g. 200 or 300) is worn by the listener. The output transducer 330 thereby receives the amplified output audio signal from the amplifier 328 and generates the playback output sounds 332, which are emitted into the ear canal when the earpiece is worn. An acoustic sum 334 including the playback output sounds 332 and the leakage sounds 306 (discussed above) is received in the inner volume of the ear canal 106 and is sensed there by the internal mic or second input transducer 336, which in the drawings (see, e.g., FIGS. 2D, 2E, 2F and 3) is illustrated as internal acoustic sensor or microphone 336. Although only a single internal microphone is illustrated in FIG. 3, it should be understood that in some embodiments the provision of one or more additional internal microphones may be beneficial (e.g., for measuring a wearer's otoacoustic emissions).

The second input transducer (internal microphone 336) is configured to be acoustically coupled to the inner volume of the ear canal when the earpiece is worn to receive the acoustic sum 334 of the playback output sounds 332 and the leakage sounds 306 and to generate an analog internal microphone audio signal 338, which in FIG. 3 is an analog signal but in an alternative embodiment may be a digital signal. In the drawings, the analog internal microphone (or second input) audio signal 338 and other signals detected by the second input transducer 336, e.g., the internal microphone, are represented in dashed lines. Signals detected by the first input transducer 308, e.g., the external microphone, are represented by sold lines.

In an example embodiment illustrated in FIGS. 3-10, the second input transducer (e.g., the internal microphone) 336 captures ear plug leakage to determine in-ear SPL, high pass filter cutoff frequency Fc, and long-term compression threshold. As discussed further below, the long-term compression threshold is used to directly reduce the output of the output transducer (e.g., the driver) 330. So the internal microphone signal 338 can be used as a control signal to generate an SPL monitor signal and/or can be used as an audio signal to generate a cancellation signal.

ADC 339 converts internal microphone audio signal 338 to a digital second input audio signal shown as 339A and 339B (in FIG. 3) or 340 (in FIGS. 4-7), in which the digitized internal microphone signals (339A, 339B and 340) are represented by dashed lines and the digitized external microphone signals (314) are represented by solid lines. Although not shown, the second input transducer (internal microphone 336) may alternatively be integrated with an ADC. The digital second input audio signal 340 can be introduced to the DSP module 316 for further processing, as discussed below.

The various connections described hereinabove and below may be wired, wireless, or a combination thereof. The wired and/or wireless connection may be used, for example, for receiving digital or audio analog signal inputs, for digital data transfer, for example, to change stored settings maintained in the memory 320 of the DSP module 316, to control operation of the processor 322, or to output data for display on a connected device, such as a smartphone. The wired connection can be, for example, one or more wires connected to a phone jack of the hearing protection system 300. The wireless connection can be provided by, for example, a Bluetooth, WiFi, or other wireless communication subsystem, module or protocol.

The DSP module 316 can include submodules represented as operational blocks for directionality processing 350, device voicing 352, corrective hearing 354, user EQ 356, ASR compression 358, ASF low frequency (LF) attenuation 360, SPL logging (e.g., internal and external microphones) 362, personal exposure metric (e.g., at the internal and external microphones) 364, and/or storage 366. The submodules are discussed in further detail below, including in connection with FIGS. 4-10. The arrangement of the operational blocks may be varied from that shown in the figures, including FIG. 3. Further, example methods described herein may involve the practice of fewer than all the operational blocks and/or practice of operational blocks not shown in the drawings.

### ASR Compression (Full Band and Multi Band)

Dynamic range compression, also referred to herein as compression, reduces or modulates the dynamic amplitude range of an audio signal over a selected frequency range.

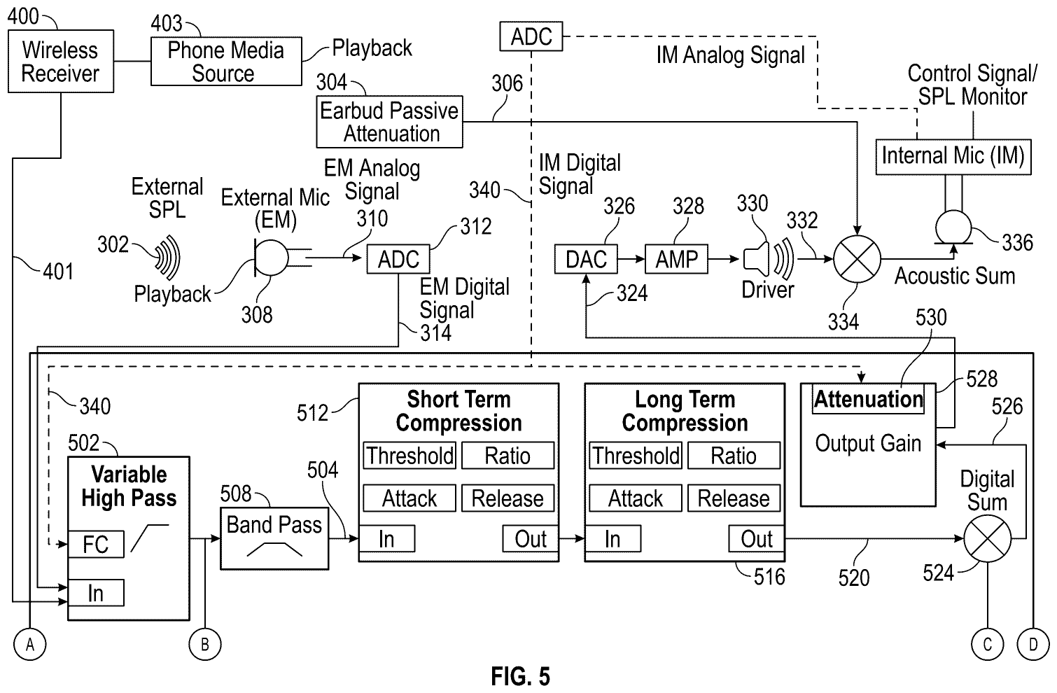

In the active hearing protection system, Active Sound Reduction (ASR) compression 358 of FIG. 3 is referred to below in separate embodiments as “full-band” ASR compression 358A in FIG. 4 and “multi-band” (e.g., dual-band or two-band) ASR compression 358B in FIG. 5. Returning to FIG. 3, ASR compression module 358 receives a processed digital signal input from external microphone 310 and a digitized input signal 339A from internal microphone 336.

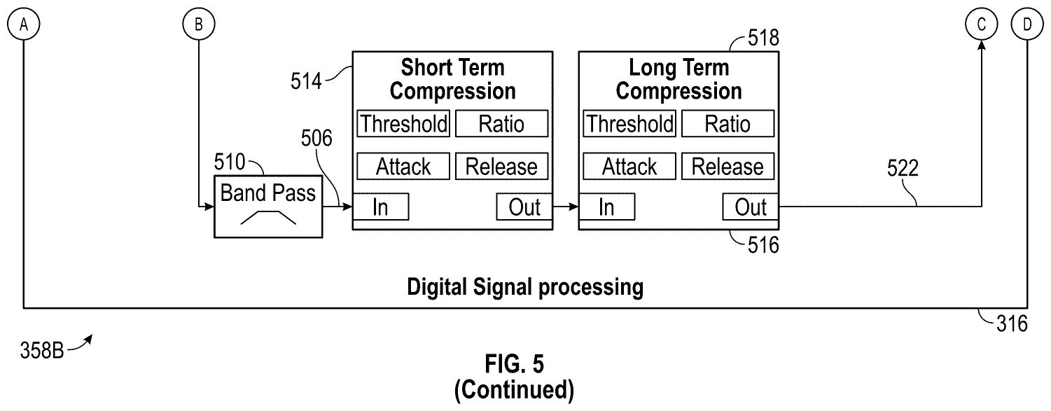

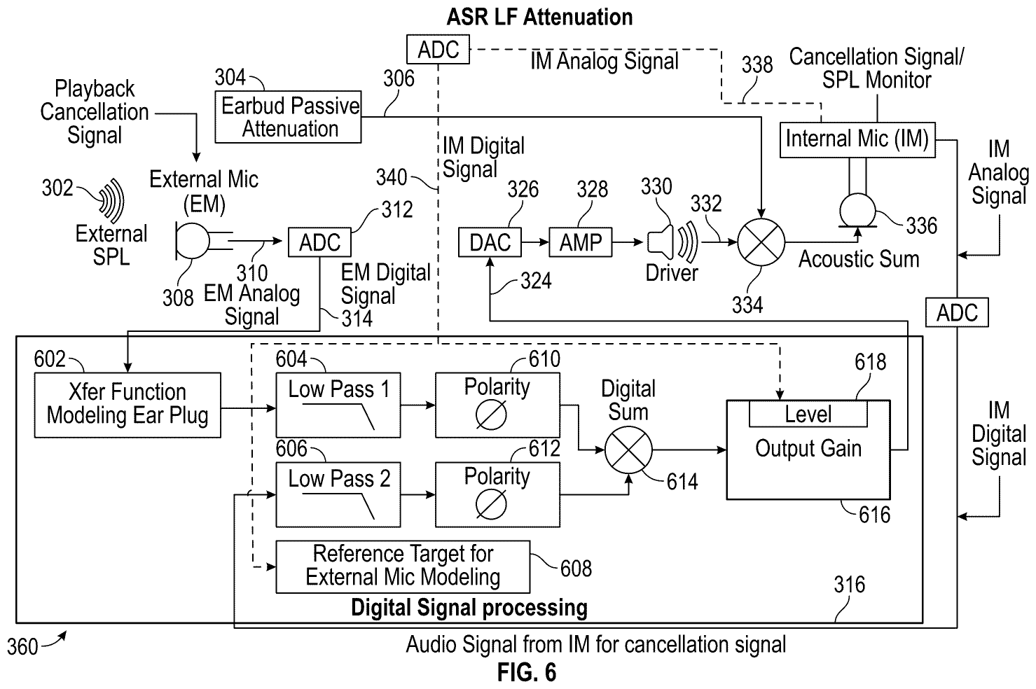

Like components, parts, and features in FIGS. 4 and 5 (as well as FIGS. 6-10 below) to those depicted in FIG. 3 are designated with like reference numerals. In the interest of simplification of description and illustration, FIGS. 4 and 5 omit depiction of certain operational blocks/submodules of the DSP module 316 of the overview schematic diagram of FIG. 3, including the submodules for directionality processing 350, device voicing 352, corrective hearing 354, user EQ 356, ASF low frequency (LF) attenuation 360, SPL logging (internal and external microphones) 362, personal exposure metric (364), and storage 366. Although not shown, those features can be included in embodiments related to FIGS. 4 and 5.

ASR compression 358 according to various embodiments disclosed herein includes short-term compression and/or long-term compression. In both short-term compression and long-term compression, the compression operation may take place over a single (or “full”) band (see FIG. 4) or may be delineated into multiple (two or more) narrower frequency bands (see FIG. 5). Either approach may be deemed more desirable in certain instances. Generally, lower frequency sounds tend to leak through the passive attenuation structure 304 more than higher frequency sounds. Further, lower frequency sounds tend to be more harmful to human ears than higher frequency sounds. For these and other reasons, it may be desirable to set different compression parameter values, such as threshold values or compression ratios, for lower frequency sounds than higher frequency sounds. For example, due to the greater harmfulness of lower frequencies, a lower band may be subject to greater compression ratios than a higher band. On the other hand, for users who are not expected to be subjected to high SPL environments often, a full band approach may be more optimal. Many of these considerations are design choices with respect to selecting full-band or multi-band operations.

In the example embodiment depicted in FIG. 4, the digital first input (external microphone) audio signal 314 and the digital second input (internal microphone) audio signal 340 are each transmitted to the DSP module 316. As explained above, certain submodules of FIG. 3 are not shown in FIG. 4 for reasons explained above, including in the interest of simplification of illustration. Nonetheless, it should be understood that in the context of FIGS. 3 and 4, the ASR compression 358A submodule (of FIG. 4) is integrated (as part of 358 of FIG. 3), so digital first input audio signal 314 and the digital second input audio signal 340 can be subject to the directionality processing 350, device voicing 352, corrective hearing 354, and/or user EQ 356, such as before being introduced to the full-band ASR compression 358A of FIG. 4, and can be further subject to the ASR LF attenuation 360 operation, such as after being subject to the full-band ASR compression 358A of FIG. 4. As described above, however, it is within the scope of the disclosure to vary the order of the operations, and/or to omit one or more of the operations illustrated in FIG. 3 and/or FIG. 4.

In FIG. 4, the digital first input audio signal 314 is introduced to a filter, such as a variable high pass 402 filter block. The variable high pass filter block 402 can selectively or variably filter or limit the lower range of frequencies that are processed and then provided to the compression blocks 404, 408. Variable high pass filter block 402 receives a digital internal microphone signal 340, such as into an “Fc” input and receives a digital external microphone input signal 314, such as into a signal input (“In”). In the example embodiment illustrated in FIG. 4 the digital second input audio signal 340 can be from external microphone 308, or it can be a playback input signal 401 (e.g., from a wireless receiver 400 and a media source 403), which are introduced into DSP module 316 directly to the variable high-pass block 402, directly to long-term compression 408 (bypassing the variable high pass 402 and short-term compression 404), and/or directly to an attenuation operation 414 (bypassing the variable high pass 402, the short-term compression 404, and the long-term compression 408). In the example embodiment illustrated in FIG. 4 the digital second input audio signal 314 can be from external microphone 308, or it can be a playback input signal 401 (e.g., from a wireless receiver 400 and a media source 403), which are introduced into DSP module 316, such as directly to the variable high-pass block 402, directly to long-term compression 408 (bypassing the variable high pass 402 and short-term compression 404), and/or directly to an attenuation operation 414 (bypassing the variable high pass 402, the short-term compression 404, and the long-term compression 408). In the example embodiment illustrated in FIG. 4 the digital second input audio signal 314 can be from internal microphone 336, which can be introduced into DSP module 316, such as directly to the variable high-pass block 402, directly to long-term compression 408 (bypassing the variable high pass 402 and short-term compression 404), and/or directly to an attenuation operation 414 (bypassing the variable high pass 402, the short-term compression 404, and the long-term compression 408).

Returning to the variable high pass 402 block or operation, the digital first input audio signal 314 and the second input audio signal 340 can thereafter transmitted to the short-term compression 404 operation and then the long-term compression operation 408 before being forwarded to an output gain 412 operation. As noted above, the digital second input audio signal 340 optionally may be introduced directly to the long-term compression 408, in which case the output gain 412 operation can follow.

Turning next to FIG. 5, which illustrates an embodiment of DSP 316 with multi-band ASR compression, the digital first input audio signal 314 and the digital second input audio signal 340 are each transmitted to the DSP module 316. As explained above, certain submodules of FIG. 3 are not shown in FIG. 5 for reasons explained above, including in the interests of brevity and simplification of illustration. Nonetheless, it should be understood that in the context of FIG. 3, of which the ASR compression 358B submodule of FIG. 5 is integrated (as 358 of FIG. 3), the digital first input audio signal 314 and/or the second input audio signal 340 can be subject to the directionality processing 350, device voicing 352, corrective hearing 354, and/or user EQ 356, such as before being introduced into the multi-band ASR compression 358B of FIG. 5 and can be further subject to the ASR LF attenuation 360, such as after being introduced into the multi-band ASR compression 358B of FIG. 5. As described above, however, it is within the scope of the disclosure to vary the order of the operations, and/or to omit one or more of the operations.

In FIG. 5, the digital first input audio signal 314 is introduced to a variable high pass 502 block or operation, which is similar to variable high pass 402, illustrated in FIG. 4 and described above. The output from variable high pass 502 can be input to a pair of Band pass filter sections 508, 510 to filter and limit the range of frequencies that are processed in the following compression sections 512, 514. In the example embodiment illustrated in FIG. 5 the digital second input audio signal 340 can be from external mic 308, or it can be a playback input signal 401 (e.g., from a wireless receiver 400 and a media source 403), which are introduced into DSP module 316 directly to the variable high-pass block 502. In the example embodiment illustrated in FIG. 5 the digital first input audio signal 314 can be from external mic 308, or it can be a playback input signal 401 (e.g., from a wireless receiver 400 and a media source 403), which can introduced into DSP module 316, such as directly to the variable high-pass block 502.

FIG. 5 depicts the digital second input audio signal 340 (as a dashed line) introduced to the variable high-pass 502 and/or directly to an attenuation operation 530 of output gain 528 block (bypassing the variable high pass 502, band passes 508 and 510, short-term compression 512 and 514, and long-term compression 516 and 518). Notably, unlike the full-band ASR compression 358A of FIG. 4, in the multi-band ASR compression 358B of FIG. 5 the digital second input audio signal 340 (originating from the internal microphone 336) does not feed directly to the long-term compression 516 in the illustrated embodiment.

Returning to the variable high pass 502 block, the digital first input audio signal 314 and the second input audio signal 340 are transmitted to a first band pass operation 508 and a second band pass operation 510 to separate the signal into a first (e.g., lower) band signal 504 fed to a first short-term compression 512 operation and a second (e.g., higher) band signal 506 fed to a second short-term compression 514 operation. The first short-term compression operation 512 with respect to first band signal 504 is associated with a first long-term compression operation 516. The second short-term compression 510 operation with respect to the second band signal 506 is associated with a second long-term compression operation 518. The signals 520 and 522 transmitted from the first and second long-term compression operations 516 and 518, respectively, can be digitally summed 524 and transmitted to an output gain block 528 which can include an attenuation control signal input that can be responsive to internal microphone digital signal 340. The resulting digital output audio signal 324 can be transmitted from the output gain operation 528 to the DAC 326, for processing as discussed above in connection with FIG. 3.

The embodiments illustrated in FIGS. 4 and 5 can both include at least one short-term compression stage and at least one long-term compression stage. In some embodiments, the ASR compression blocks (e.g., of FIGS. 4-7) can be “mid-tier” and that ASR can be for low frequency attenuation; those two things can work in tandem to execute the automatic sound reduction (“ASR”) algorithm which addresses the bulk of the environmental noise—of sound reduction that this system is designed to process and then (in FIG. 3) SPL logging block 362 can be also capturing recordings over time and logging or recording the user's noise dosage (e.g., noise the user is exposed to throughout the day or over time) and based on that SPL logging data, the Hearing Protection System 300 may do some further attenuation of the signal to make sure the user is not exceeding pre-selected upper limits on noise exposure dosages. It's worth noting here that System 300 can accumulate user exposure or noise dosage data in real time and in response attenuate sounds for that user's then extant hearing protection needs, not based solely on the strength of the current environmental sound 302, but also on the user's recent history. For example, if the user is present at a loud event with a lot of people and fireworks start going off; a signal like that (that is very loud and impulsive would) can very quickly trigger the compressors and protect the user/listener from damaging effects of a really loud impulsive sound. Then there's also more long-term environmental sounds like going to a rock concert where there is continuous or substantially constant excessively loud sounds such as at excessively high Sound Pressure Levels (“SPLs”). In response to the continuously excessive SPLs, the system can squeeze or compress that signal and playback the signal at a lower, safe level from driver 330. If the user is at a festival all day long, they'll likely hit a threshold (e.g., an 8-hour cap) of exceeding noise exposure, and then system 300 can start to provide further reductions to comply with longer term exposure (e.g., OSHA) standards.

The user's SPL logging data can track how long the user has been exposed to high SPLs over the day (or other time period) and then eventually that can factor into what is also being played back so the user-specific Hearing Protection will refine itself through the day (or time period). The noise exposure protection standards do not have to be from a government regulation. Protection could be adjusted in response to an audiologist's advice (e.g., recommending that the user only be exposed to 72 dB maximum for 8 hours a day) which can be entered into an associated system or application, e.g. that both the user and the audiologist can review as part of an audiological care plan, whereby system 300 can enable the user and their audiologist to execute, monitor and update that plan in real time, and remotely in some cases.

Generally, short-term compression refers to sound reduction to the digital first input audio signal 314 and/or the digital second input audio signal 340 in the short term, such as in transient or real-time (substantially instantaneous) without consideration of exposure history. For example, and without limitation, if the wearer of the system 300 experiences transient high SPL, such as while attending a firearm shooting match, concert or sporting event, the system 300 can react to high SPL by providing sound reduction (e.g., in substantially real time) irrespective of exposure history. According to an example embodiment, short-term compression involves reduction of high amplitude transients.

On the other hand, long-term compression generally refers to sound reduction based upon long-term exposure of the ear canal. In example embodiments, the long-term exposure is determined based upon, at least, an acoustic sum of the playback output sounds 332 and the leakage sounds 306 based on measurements over a selected period of time. According to an example embodiment, long-term compression involves long-term monitoring of a digital input audio signal, in some embodiments the second audio input signal 340, transmitted to the DSP module 316. The second input audio signal 340 reflects the acoustic sum 334 of the playback output sounds 332 and the leakage sounds 306 detected by the internal microphone 336. Values of the second input audio signal 340 can be logged to memory 322, 323 over time to accumulate the user's exposure history as sensed within the ear canal 106 to an acoustic sum of the playback output sounds and the leakage sounds.

Short-term and long-term compression can be controlled by selecting certain compression parameters. According to an example embodiment, those parameters include one or more of compression threshold, threshold ratio, compression attack, and compression release. Compression threshold refers to amplitude, often in decibels, at which the compressor is activated to reduce the amplitude of an audio signal. When a signal level is below a selected audio signal amplitude threshold, no compression is performed, whereas compression is performed when the signal level reaches or exceeds the threshold. Threshold ratio refers to the amount of gain reduction provided via compression. For example, a ratio of 5:1 means that for an input level of 4 dB over the threshold, the output signal level is reduced to 1 dB over the threshold. Compression attack and release reflect the degree of control over how quickly a compressor acts initially and then how quickly it ceases. The threshold attack refers to the period when the compressor is decreasing gain in response to the input to reach the gain determined ratio. The threshold release refers to the period of time when the compressor is restoring or increasing gain in response to reduced level at the input to reach the output gain determined by the ratio, or to unity, once the input level has fallen below the threshold.

Non-limiting examples of parameters and values for carrying out active sound reduction (ASR) full-band compression (e.g., FIG. 4) and multi-band compression (e.g., FIG. 5) are set forth below in TABLES 1 and 2, respectively. Generally, the variable high Pass Fc, the short-term and long-term compressor threshold, the short-term and long-term compressor ratio, the short-term and long-term compressor attack, the short-term and long-term release, and the gain attenuation of TABLE 1 are identical to those values in TABLE 2, although in some cases different values or ranges can be used for ASR full-band compression than for ASR multi-band compression. For the multi-band compressor block of TABLE 2, the first bandpass and the second bandpass are set at values of 1-2 KHz and 2-4 kHz, respectively.

TABLE 1
(ASR Full-Band Compressor Block
Example Parameters and Values)

| Parameter | Value |
|---|---|
| Variable Hi Pass Fc | 20 Hz to 5 kHz |
| Short-Term Compressor Threshold | 0 dBF to −40 dBF |
| Short-Term Compressor Ratio | 10:1 or higher |
| Short-Term Compressor Attack | <1 ms |
| Short-Term Compressor Release | 100 ms |
| Long-Term Compressor Threshold | 0 dBF to −40 dBF |
| Long-Term Compressor Ratio | 5:1 |
| Long-Term Compressor Attack | <20 ms |
| Long-Term Compressor Release | 500 ms to 1000 ms |
| Gain Attenuation | 0 to −30 dB |

TABLE 2
(ASR Multi-Band Compressor Block
Example Parameters and Values)

| Parameter | Value |
|---|---|
| Variable Hi Pass Fc | 20 Hz to 5 kHz |
| Short-Term Compressor Threshold | 0 dBF to −40 dBF |
| Short-Term Compressor Ratio | 10:1 |
| Short-Term Compressor Attack | <1 ms |
| Short-Term Compressor Release | 100 ms |
| Long-Term Compressor Threshold | 0 dBF to −40 dBF |
| Long-Term Compressor Ratio | 5:1 |
| Long-Term Compressor Attack | <20 ms |
| Long-Term Compressor Release | 500 ms to 1000 ms |
| Bandpass | 1 kHz to 2 kHz |
| Bandpass | 2 kHz to 4 kHz |
| Gain Attenuation | 0 to −30 dB |

TABLE 2 sets forth two bands of 1-2 kHz and 2-4 kHz. It is possible to select three or more bands for the multi-band compression, and hence three or more short-term compression operations and three or more long-term compression operations (e.g., thus adding another short-term compression operation in addition to the two short-term compression operations 512 and 514 and adding another long-term compression operation in addition to the two long-term compression operations 516 and 518 of FIG. 5). For example, and without limitation, an example of a three-band compression may involve a first bandpass of 1-2 kHz, a second bandpass of 2-3 kHz, and a third bandpass of 3-4 kHz. The bands may extend below 1 kHz and above 4 kHz. Other suitable frequency bands can be used.

As explained above, short-term compression can be monitored and controlled substantially in real-time to control transient high-level sound pressure. If the SPL of the analyzed signal 314 and/or 340 exceeds the compression threshold, short-term compression can be initiated.

On the other hand, long-term compression is based upon accumulated exposure history of the ear canal to an acoustic sum of the playback output sounds and the leakage sounds over a period of time to control non-transient high-level sound pressure. The period of time for monitoring may be, for example and without limitation, five (5) minutes, any increment within a range of 5-60 minutes, one (1) hour, more than one hour, multiple hours, twenty-four (24) hours, any hourly increment between one (1) hour and twenty-four (24) hours (e.g., eight (8) hours or a work period), more than twenty-four (24) hours, or any number of days, e.g., two days, three days, etc.

The automated safe hearing algorithm(s) (such as the long-term compression operations) can be configured to reduce SPL in the listener's ear canal(s) to recognized safe levels of dosage over time according to an example embodiment. In an example embodiment, long-term compression attenuates root-mean-square (RMS) levels over a period of time from exceeding a safe dosage limit.

According to an embodiment, SPL exposure at the ear of any frequency or an average of frequencies is determined as the external SPL minus any attenuation plus any corrective gain. Attenuation can include compression, parametric equalization, filtering, destructive interference, and any embodiment of ASR. Corrective gain is the increase in gain to compensate for hearing loss (HL) (e.g., found from the OAE test). The microphone-measured SPL may be unweighted, A-weighted, or use a different weighting.

According to an embodiment, SPL exposure is determined using the following equation (1) for a single number average, wherein N is the number of frequency points:

$$= ( 1 / N ) * \sum [ ( SPL at external mics ) - ( attenuation ) + ( corrective gain ) ] \qquad ( 1 )$$

According to another operation, SPL exposure is determined at each frequency across the total frequency spectrum under analysis, e.g., 20 Hz to 20 kHz, using equation (2) below:

$$\sum [ ( SPL at external mics ) - ( attenuation ) + ( corrective gain ) ] \qquad ( 2 )$$

Various other suitable equations or formulas can be used to determine SPL exposure. According to an embodiment, SPL exposure can be a product, average, or other value calculated from any combination or all of the above operations.

Examples of long-term exposure safety standards that optionally may be used to define the period of time for the exposure history are provided by the Occupational Safety and Health Administration, also known as OSHA. The standards may be used in example embodiments to determine compression thresholds. An example of OSHA recommended SPL dosage limits is provided in TABLE 3 below:

TABLE 3
(OSHA Recommended SPL Dosage Limits)

| Sound Level (dBA) | Permitted Duration Per Workday (Hours) |
|---|---|
| 90 | 8.00 |
| 91 | 6.96 |
| 92 | 6.06 |
| 93 | 5.28 |
| 94 | 4.60 |
| 95 | 4.00 |
| 96 | 3.48 |
| 97 | 3.03 |
| 98 | 2.63 |
| 99 | 2.30 |
| 100 | 2.00 |
| 101 | 1.73 |
| 102 | 1.52 |

For example and without limitation, a long-term exposure standard may involve limiting a user to an average of no more than 90 dBA over an 8-hour period, or 100 dBA over a 2-hour period (see TABLE 3). It should be understood that OSHA standards are given as examples, and that other standards and values may be applied in accordance with embodiments described herein, e.g., a 72 dBA maximum for an 8-hour period. For example, long-term compression parameters and values may be developed by an audiologist or other physician as part of a hearing profile.

A non-limiting example of an order of operations for full-band and multi-band compression is provided below:
1. Turn on device.
2. Internal microphone senses the leakage level, sets high pass Fc, long-term compressor threshold, and/or output gain attenuation.
3. External microphone signal is fed through the two-stage (short-term and long-term) compressor (full-band or multi-band).
4. A compressed signal is played back through the driver/speaker.
5. The Total Sound Pressure Level (SPL) is equal to the sum of the driver output and the ear plug leakage, with the summing at the internal microphone (also the ear drum).
6. The Total SPL is continually monitored by the internal microphone. If a safe level is exceeded, long-term threshold is reduced.
7. In the case of multi-band compression, in some embodiments the internal microphone does not feed the long-term compression threshold, but feeds the output gain attenuation.

A non-limiting example of conditions of compression for full-band and multi-band compression is provided below:
1. Based on ear plug leakage amplitude, the filter cutoff frequency Fc of the HP filter is adjusted accordingly, e.g., external sound of 100 dB would have a low Fc and external sound of 120 dB would have a high Fc. A higher intensity can result in a higher Fc and a lower intensity can result in a lower Fc.
2. Based on the ear plug leakage amplitude, the long-term compressor threshold or output gain attenuation is adjusted, such as to achieve playback levels that are, for example, less than or equal to 85 dB SPL. For example, for an SPL at the internal microphone of less than or equal to 85 dB SPL, the playback is equivalent to the environment; for an SPL at the internal microphone of more than 85 dB SPL, the playback is set at 85 dB SPL at the ear canal; and for an SPL at the internal microphone equal to 85 dB SPL for more than 8 hours, playback is less than 85 db SPL at the ear canal. According to an embodiment, the goal is to satisfy the noise dosage levels in compliance with current (e.g., OSHA) standards.

### ASR Low Frequency Attenuation

As discussed above, lower frequencies have a greater tendency to leak through the passive attenuation structure 304. Further, lower frequencies are more harmful to human ears than higher frequencies. FIG. 6 illustrates an embodiment of ASR low frequency (LF) attenuation 360.

The ASR LF attenuation 360 constitutes part of the flow diagram of FIG. 3, discussed above. As explained above, certain submodules of FIG. 3 are not shown in FIG. 6 for reasons explained above, including in the interests of brevity and simplification of illustration. Nonetheless, it should be understood that in the context of FIG. 3, of which the ASR LF attenuation 360 submodule of FIG. 6 is integrated, the digital first input audio signal 314 and the second input audio signal 340 can be subject to the directionality processing 350, device voicing 352, corrective hearing 354, user EQ 356, and/or ASR compression 358, such as before being introduced to the ASR LF attenuation 360 of FIG. 6. As described above, however, it is within the scope of the disclosure to vary the order of the operations, and/or to omit one or more of the operations.

In FIG. 6, the digital first audio input signal 314 is transmitted to a Xfer function modeling ear plug 602 (e.g., a transfer function), followed by a low pass 1 operation 604 and a polarity operation 610 (e.g., polarity inversion). The digital second input audio signal 340 is transmitted to a low pass operation 606, a reference target for external microphone modeling operation 608, and a level operation 618. A polarity operation 612 (e.g., polarity inversion) follows the low pass operation 606. Output signals from the polarity operations 610 and 61 can be digitally summed 614 and can be subject to an output gain operation 616.

A non-limiting example of parameters and values for carrying out active sound reduction (ASR) low frequency (LF) attenuation (e.g., FIG. 6) is set forth below in TABLE 4.

TABLE 4
(ASR LF Attenuation Block Example Parameters and Values)

| Parameter | Value |
|---|---|
| Low Pass 1 Filter Fc | 100 Hz to 200 Hz |
| Low Pass 2 Filter Fc | 100 Hz to 200 Hz |
| Output Gain Level | 0 to −30 dB |

A non-limiting example of an order of operations for ASR LF attenuation is provided below:
1. If the external SPL overwhelms passive attenuation of the ear plug (e.g., the passive attenuation structure 304) as determined by the internal microphone 336 logging SPL, the LF attenuation processing 360 engages.
2. According to an example embodiment, the following are applied: (a) a low pass filter, (b) polarity inversion, and (c) variable gain such as between −12 dB and 0 dB.
3. Playback through the output transducer (e.g., the driver) 330 acts as a destructive signal to the ear plug leakage.

FIG. 11 illustrates an example of an ASR LF attenuation simulation, showing the signal “as is” (i.e., without attenuation) and the signal conditioned with LF ASR.

A non-limiting example of conditions of ASR LF protection are set forth below:
1. ASR LF protection is engaged (in an example embodiment) when very high SPL, low-frequency signals have exceeded the protection capabilities of the passive attenuation structure of the earpiece.
2. When the external SPL (full spectrum) hits 120 dB, the eardrum will be exposed to approximately 86 dB with foam tips, for example. This level is at the edge of safe limits for a given exposure time, in some cases.
3. When active attenuation reaches its limits, any further increases in SPL can be beyond the device's Active Sound Reduction capability, in some circumstances.

In an embodiment, ASR LF involves automatically alternating for the listener's application needs in real time, with the alternating taking place between active sound reduction and active noise cancellation (ANC). In an embodiment, active sound reduction involves high-fidelity low distortion compression, high frequency environment reproduction (feedforward/feedback compression and gain), low frequency attenuation (hybrid; feedforward and feedback), and/or conservation of hearing. In an embodiment, active noise cancellation involves high-fidelity, low distortion music and media reproduction, clear and intelligible telecommunications, and hybrids (combination of feedforward and feedback).

Actuation of the ANC feature can be determined by measure the SPL of leakage sounds 306 that bypass the passive attenuation structure 304, as measured by the internal microphone 336. In an embodiment, ANC is initiated when the leakage sounds 306 (as calculated based on measurements by the internal microphone 336) exceed a threshold.

### Directionality Processing

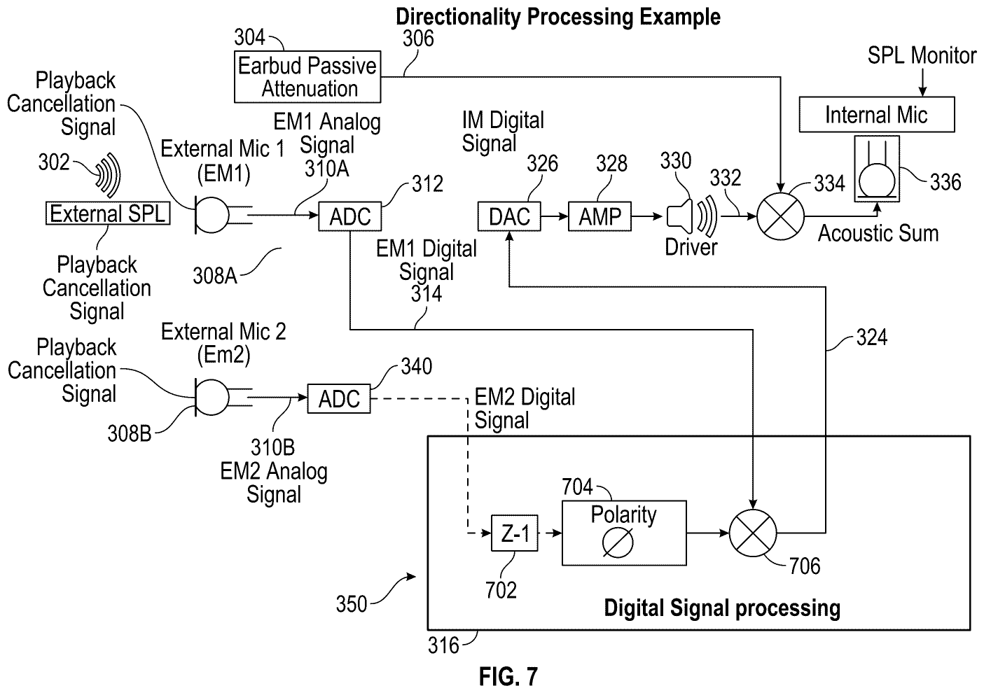

FIG. 7 illustrates an embodiment of directionality processing 350. The directionality processing 350 constitutes part of the flow diagram of FIG. 3, discussed above. As explained above, certain submodules of FIG. 3 are not shown in FIG. 7 for reasons explained above, including in the interests of brevity and simplification of illustration. Forward and rearward and/or left and right side directional cues may be generated, such as in a two ear embodiment of the system. A two-ear system would include at least a first or left external microphone 308 for a first or left earpiece (or earbud) and a second or right external microphone for a second or right earpiece (or earbud). Alternatively, one earpiece (or earbud) or both could carry multiple (e.g., front and rearwardly aimed) directional external microphones and directionality processing circuitry 350 to provide improved directionality operations.

Nonetheless, it should be understood that in the context of FIG. 3, of which the directionality processing 350 submodule of FIG. 7 is integrated, the digital first input audio signal 314 and the second input audio signal 340 can be subject to the device voicing 352, corrective hearing 354, user EQ 356, ASR compression 358, and/or ASR LF attenuation 360, such as after being introduced to the directionality processing 350 of FIG. 7. As described above, however, it is within the scope of the disclosure to vary the order of the operations, and/or to omit one or more of the operations.

In an example embodiment, the system 300 includes two or more external audio input transducers, such as two or more microphones, to facilitate directionality measurements. Directionality can function to, for example, assist in determining the positioning of the wearer. In another embodiment, directionality can be used to focus on sounds in front of (as opposed to behind) the wearer of the system 300. The system can determine a direction of the sound, such as based on arrival times at the different microphones, and the system can determine an amount of compression or attenuation or amplification based on the direction of the sound (e.g., the direction relative to the wearer or relative to the earpiece). In some cases, sound from behind the user can be attenuate or compressed more than sound in front of the user. For example, a cardioid pattern could be achieved to reject sound from behind the user.

Turning to FIG. 7, first and second external microphones 308A and 308B are carried in the housing 206 and each generates an analog audio signal which is digitized to generate an EM1 digital signal 310A and an EM2 digital signal 310B which are input to the directionality processing module 350 (e.g., within DSP module 316). Directionality processing module 350 can include a Z-1 block 702 which receives EM2 digital signal and generates a signal which is input to polarity block 704, that generates a signal (e.g., inverted signal) for input to a mixing or summing block that also receives EM1 digital signal 314 to generate a processed output signal 324. In the example embodiment illustrated in FIG. 7 the digital input audio signals can be from external mics 308A and 308B, or can be a playback input signals (e.g., from a wireless receiver 400 and a media source 403), which are introduced into DSP module 316. Although FIG. 7 shows two external microphones, some implementations can use additional microphones.

### Device Voicing

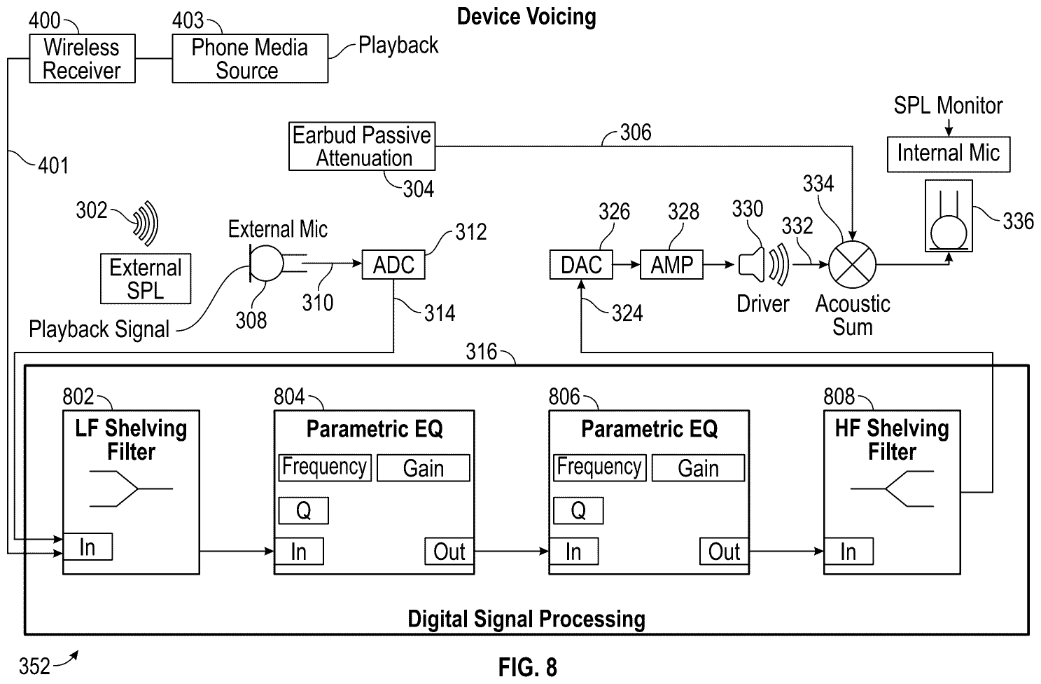

FIG. 8 illustrates an embodiment of device voicing 352. The device voicing 352 operation constitutes part of the flow diagram of FIG. 3, discussed above. As explained above, certain submodules of FIG. 3 are not shown in FIG. 8 for reasons explained above, including in the interests of brevity and simplification of illustration. Nonetheless, it should be understood that in the context of FIG. 3, of which the device voicing 352 submodule of FIG. 8 is integrated, the digital first input audio signal 314 can be subject to the directionality processing 350 before being introduced into the device voicing 352 and can be further subject to the corrective hearing 354, user EQ 356, ASR compression 358, and ASR LF attenuation 360, such as after being introduced to the device voicing 352 of FIG. 8. As described above, however, it is within the scope of the disclosure to vary the order of the operations, and/or to omit one or more of the operations.

In FIG. 8, the digital first input audio signal 314 is transmitted to a low frequency (e.g., LF shelving) filter (802), followed by a first parametric EQ operation 804 and a second parametric EQ operation 806, followed by a high frequency (HF) filter (e.g., shelving filter) operation 808. In the example embodiment illustrated in FIG. 8 the digital input audio signal can be from external microphone 308, or it can be a playback input signal 401 (e.g., from a wireless receiver 400 and a media source 403), which can be introduced into DSP module 316 directly to the LF shelving filter 802. Many variations are possible. For example, in some implementations as single parametric EQ operation can be used.

A non-limiting example of parameters and values for carrying out device voicing (e.g., as illustrated in FIG. 8) is set forth below in TABLE 5.

TABLE 5
(Device Voicing Block Example Parameters and Values)

| Parameter | Value | Comment |
|---|---|---|
| LF Shelving Filter Gain | −12 dB to +6 dB | |

| | | | |
|---|---|---|---|
| LF Shelving Filter Fc | 150 | Hz | |
| PEQ1 Fc | 3.5 | kHz | HRTF Target |
| PEQ1 Gain | 10 | dB | |

| | |
|---|---|
| PEQ1 Q | 4 |

| | | | |
|---|---|---|---|
| PEQ2 Fc | 10 | kHz | HRTF Target |
| PEQ2 Gain | 5 | dB | |

| | |
|---|---|
| PEQ2 Q | 4 |
| HF Shelving Filter | −12 dB to +6 dB |

| | | |
|---|---|---|
| HF Shelving Filter Fc | 2 | kHz |

### User EQ

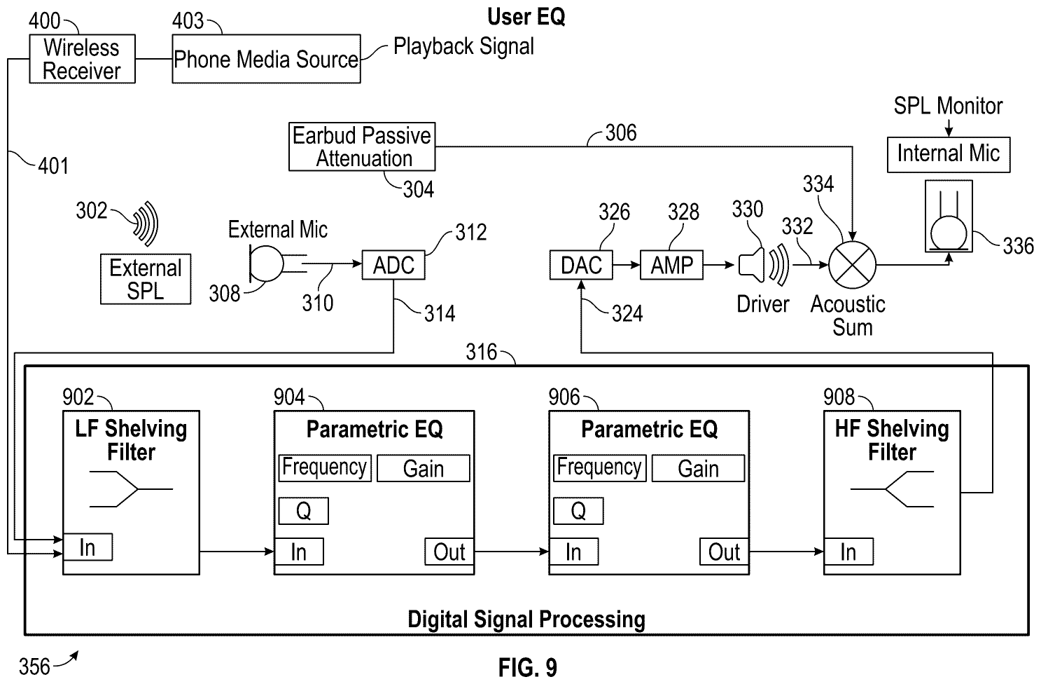

FIG. 9 illustrates an embodiment of User EQ 356. The User EQ 356 operation constitutes part of the flow diagram of FIG. 3, discussed above. As explained above, certain submodules of FIG. 3 are not shown in FIG. 9 for reasons explained above, including in the interests of brevity and simplification of illustration. Nonetheless, it should be understood that in the context of FIG. 3, of which the User EQ 356 submodule of FIG. 9 is integrated, the digital first input audio signal 314 can be subject to the directionality processing 350, device voicing, and corrective hearing 354, such as before being introduced into the User EQ 356 operation, and can be further subject to the ASR compression 358 and ASR LF attenuation 360, such as after being introduced to the User EQ 356 operation of FIG. 9. As described above, however, it is within the scope of the disclosure to vary the order of the operations, and/or to omit one or more of the operations.

In FIG. 9, the digital first input audio signal 314 is transmitted to a low frequency filter (e.g., LF shelving filter) 902, followed by a first parametric EQ operation 904 and a second parametric EQ operation 906, followed by a high frequency (HF) filter (e.g., HF shelving filter) operation 908. In the example embodiment illustrated in FIG. 9 the digital input audio signal can be from external mic 308, or it can be a playback input signal 401 (e.g., from a wireless receiver 400 and a media source 403), which are introduced into DSP module 316 directly to the LF shelving filter 902. Many variations are possible. For example, in some implementations as single parametric EQ operation can be used.

The corrective EQ processing can be independent from the User EQ processing.

Non-limiting examples of parameters and values for carrying out User EQ (e.g., FIG. 9) are set forth below in TABLE 6.

TABLE 6
(EQ Block Example Parameters and Values)

| Parameter | Value | |
|---|---|---|
| LF Shelving Filter Gain | −12 dB to +6 dB | |
| LF Shelving Filter Fc | 150 | Hz |
| PEQ1 Fc | 3.5 | kHz |
| PEQ1 Gain | 10 | dB |
| PEQ1 Q | 4 | |
| PEQ2 Fc | 10 | kHz |
| PEQ2 Gain | 5 | dB |
| PEQ2 Q | 4 | |
| HF Shelving Filter | −12 dB to +6 dB | |
| HF Shelving Filter Fc | 2 | kHz |

### SPL Logging

A discussion of SPL logging corresponding to block 362 of FIG. 3 according to an example embodiment follows.

Automated Safe Hearing Algorithm: SPL logging 362 logs SPL dosage over time for the user. In this regard, the SPL logging can be similar to a wearable fitness tracker (e.g., a Fitbit™), but is directed to logging data relating to sound.

Non-limiting examples of conditions for SPL logging are provided below:
1. SPL at the external microphone 308 of less than or equal to a threshold (e.g., 85 dB) SPL, playback is equivalent to the environment;
2. SPL at microphone 308 of more than a threshold (e.g., 85 dB) SPL, playback is set to a value (e.g., 85 dB) SPL at the ear canal 106; and
3. SPL at the microphone 308 equal to an exposure level (e.g., 85 dB SPL for more than 8 hours), playback is set to a lower SPL value (e.g., less than 85 dB SPL) at the ear canal 106.

According to an embodiment, the goal is to satisfy the noise dosage levels in compliance with current, desired, or prescribed standards.

According to an embodiment, the SPL logging involves continuous recording of data at the first input transducer 308 and the second input transducer 336, e.g., the internal and external microphones, respectively. In an example embodiment, independent (discrete) data is recorded for the left ear and the right ear. The data may be logged, for example, in the storage 323 or the memory 322.

In an example embodiment, the user or other person can review the data, such as using a smartphone, tablet, or other computer connected (wired or wirelessly) to the system 300. According to an example embodiment, the system 300 provides an alert (e.g., noise, visual signal, etc.) to the user or other person (e.g., an audiologist) monitoring the system 300 in a high SPL environment. The alert may serve, for example, to recommend that the user leave or limit their exposure to the high SPL environment. According to another example embodiment, logged data (e.g., the SPL data), parameters and values, and/or periodic audiograms are transmitted to the user or another person (e.g., an audiologist). Optionally, the transmission is conducted over a computer network, such as a LAN or the Internet.

Various conclusions and information may be drawn from the recording and reporting of SPL data from the first and second input transducers 308 and 336, respectively. For example, a difference between the values recorded at the respective transducers 308 and 336 might suggest a poor seal by the earpiece, in particular the passive attenuation structure 304. Such information can be used to flag or otherwise alert the user or other person (e.g., an audiologist) monitoring the system 300 that the seal is not proper and should be improved or corrected, such as by reshaping the earpiece to improve the seating of the earpiece in the ear to seal the ear canal.

### Personal Exposure Metric

A discussion of corrective SPL exposure corresponding to block 364 of FIG. 3 according to an example embodiment follows.

According to an example embodiment, personal exposure metric 364, exposure to SPL is measured at the first and second input transducers 308 and 336, respectively, e.g., the external and internal microphones, respectively.

According to an embodiment, personal exposure metric 364 includes consideration of the user's otoacoustic emissions (OAE) or distorted-product otoacoustic emissions (DP-OAE). A discussion of OAE and DP-OAE are set forth in U.S. Pat. No. 10,708,680 entitled “Personalization of Auditory Stimulus,” which is incorporated by reference herein for all that it discloses.

According to a first operation, SPL exposure at the ear of any frequency or an average of frequencies is determined as the external SPL minus any attenuation plus any corrective gain. Attenuation can include compression, parametric equalization, filtering, destructive interference, and/or any embodiment of ASR. Corrective gain is the increase in gain to compensate for hearing loss (HL), such as found from the OAE test. The microphone-measured SPL may be unweighted, A-weighted, or use a different weighting.

According to a second operation, SPL exposure is determined using the following equation (1) for a single number average, wherein N is the number of frequency points:

$$= ( 1 / N ) * \sum [ ( SPL at external mics ) - ( attenuation ) + ( corrective gain ) ] \qquad ( 1 )$$

According to a third operation, SPL exposure is determined at each frequency across the total frequency spectrum under analysis, e.g., 20 Hz to 20 kHz, using equation (2) below:

$$\sum [ ( SPL at external mics ) - ( attenuation ) + ( corrective gain ) ] \qquad ( 2 )$$

According to an embodiment, SPL exposure can be a product, average, or other value calculated from any combination or all of the above operations.

According to an embodiment, otoacoustic measurements are taken periodically and a rate of change over time for the user is monitored. According to an embodiment, the otoacoustic measurements are compared to other users, for example anonymously. According to an embodiment, the otoacoustic measurements may be sent to an audiologist, otolaryngologist, or other physician or third person.

### Corrective Hearing

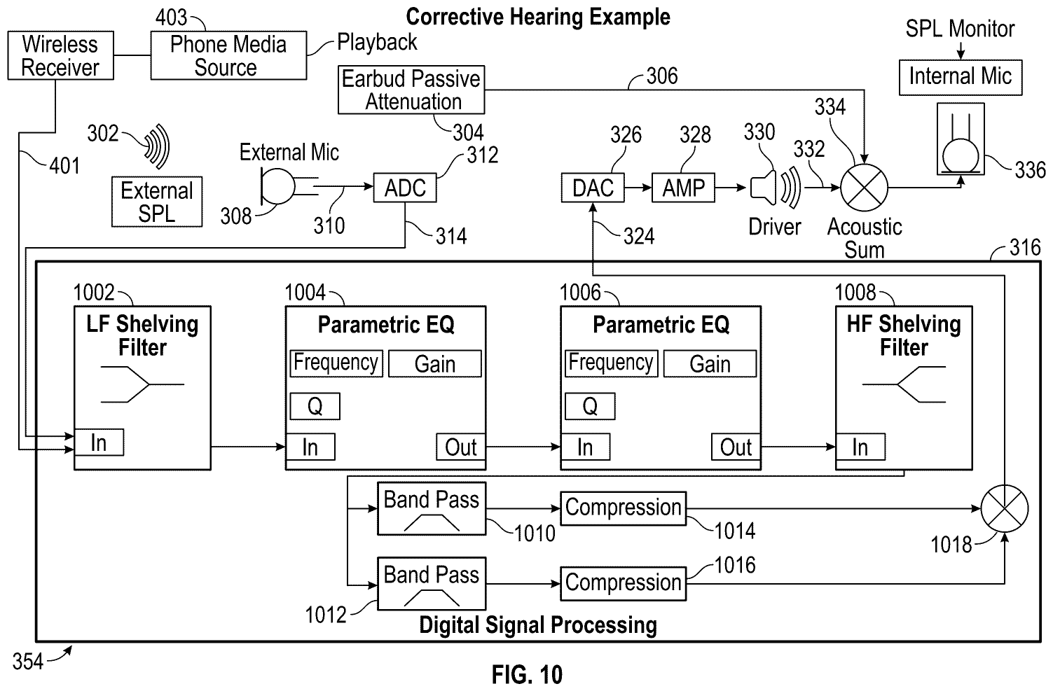

FIG. 10 illustrates an embodiment of the DSP 316 incorporating corrective hearing processing 354. The corrective hearing 354 operation constitutes part of the flow diagram of FIG. 3, discussed above. As explained above, certain submodules of FIG. 3 are not shown in FIG. 10 for reasons explained above, including in the interests of brevity and simplification of illustration. Nonetheless, it should be understood that in the context of FIG. 3, of which the corrective hearing 354 submodule of FIG. 10 is integrated, the digital first input audio signal 314 can be subject to the directionality processing 350 and device voicing 352, such as before being introduced to the corrective hearing 354 operation and can be further subject to the User EQ 356, ASR compression 358, and ASR LF attenuation 360, such as after being introduced to the corrective hearing 354 operation of FIG. 10. As described above, however, it is within the scope of the disclosure to vary the order of the operations, and/or to omit one or more of the operations.

A non-limiting example of parameters and values for carrying out corrective hearing (e.g., FIG. 10) is set forth below in TABLE 7.

TABLE 7
(Corrective Hearing Block Example Parameters and Values)

| Parameter | Value | Comment |
|---|---|---|
| LF Shelving Filter Gain | −12 dB to +6 dB | |

| | | | |
|---|---|---|---|
| LF Shelving Filter Fc | 150 | Hz | |
| PEQ1 Fc | 3.5 | kHz | HRTF Target |
| PEQ1 Gain | 10 | dB | |

| | |
|---|---|
| PEQ1 Q | 4 |

| | | | |
|---|---|---|---|
| PEQ2 Fc | 10 | kHz | HRTF Target |
| PEQ2 Gain | 5 | dB | |

| | |
|---|---|
| PEQ2 Q | 4 |
| HF Shelving Filter | −12 dB to +6 dB |

| | | |
|---|---|---|
| HF Shelving Filter Fc | 2 | kHz |

| | |
|---|---|
| Bandpass | 1 kHz to 2 kHz |
| Bandpass | 2 kHz to 4 kHz |

Other potential applications of embodiments and broader teachings of corrective hearing disclosed herein include:
1. Active sound reduction involving a combination of high frequency reproduction, low frequency attenuation, and transfer function of passive attenuation.
2. SPL dosage logging in conjunction with an “in-ear” device that utilizes an active sound reduction algorithm.
3. Periodic otoacoustic audiogram logging that analyzes hearing over time, including logging data made available to audiologists, such as over the cloud.

### Graphs

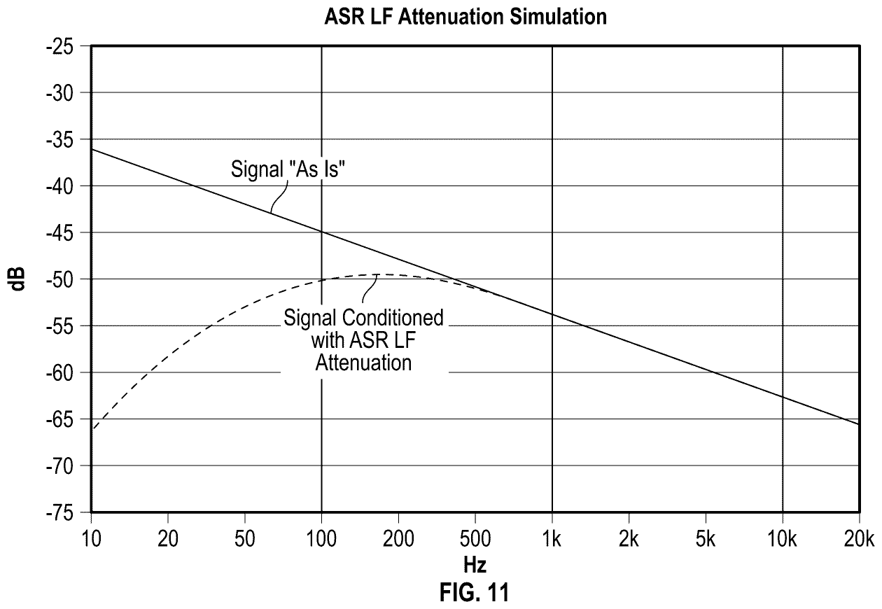

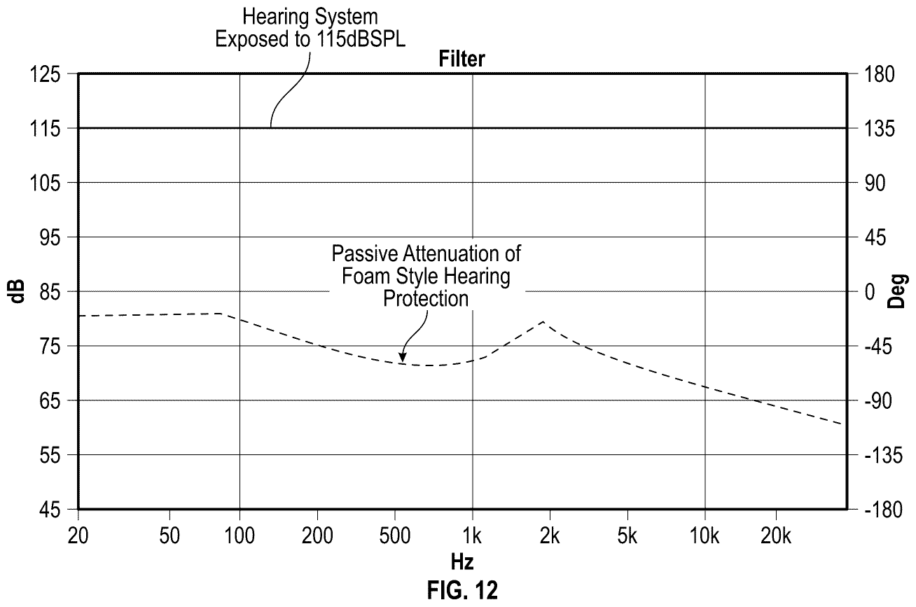

FIG. 12 is a graph illustrating sound reduction for a passive attenuation, foam-style hearing protection system exposed to 115 dB environmental SPL, wherein the horizontal axis represents frequency (Hz, starting at 20 Hz) and the left-side vertical axis represents SPL in decibels (dB). The upper plot is linear and represents the hearing protection system exposed to 115 dB SPL. Passive attenuation of the foam-style hearing protection is demonstrated by the lower plot, which is non-linear. Generally, FIG. 12 shows that passive attenuation of the foam-style hearing protection can, in some circumstances, provide adequate hearing protection. At the same time, however, in the example of FIG. 12, the high frequencies “roll off,” which degrades the fidelity of the signal.

FIG. 13 is a graph illustrating sound reduction for a high frequency reproduction hearing protection system exposed to 115 dB environmental SPL, i.e., the same exposure as in FIG. 12, wherein the horizontal axis represents frequency (Hz, starting at 20 Hz) and the left-side vertical axis represents SPL in decibels (dB). The additional plotted line in FIG. 13 (not shown in FIG. 12) is for high frequency reproduction. The sound pressure level (SPL) of the high frequency reproduction exceeds that of the passive attenuation at a relatively high frequency of about (slightly less than) 4,000 Hz and greater frequencies. The high frequency reproduction can be implemented by the system, such as by receiving the environmental sounds at the external microphone, and replaying a frequency band using the driver of the earpiece.

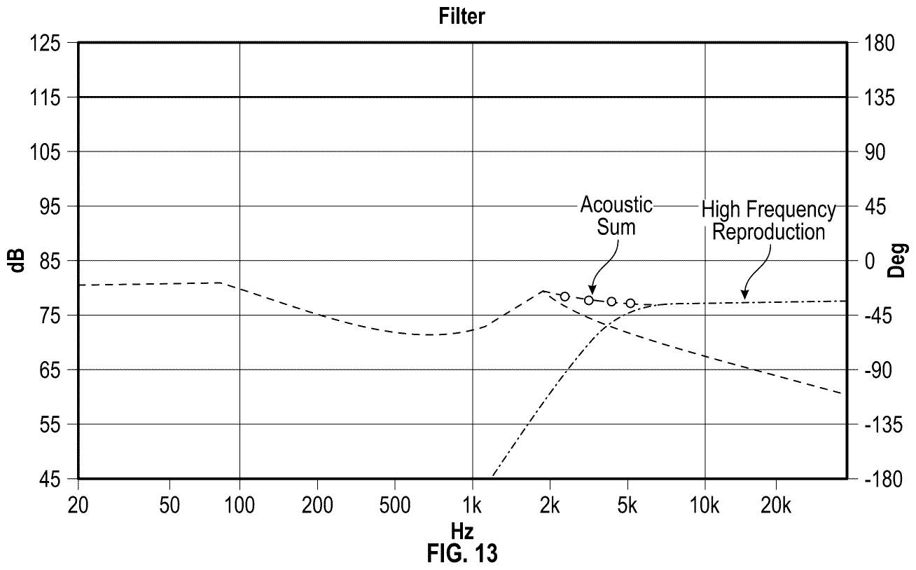

FIG. 14 is a graph illustrating active sound reduction (ASR) for a hearing protection system according to an example embodiment exposed to 115 dB environmental SPL (i.e., the same SPL as FIGS. 12 and 13), wherein the horizontal axis represents frequency (Hz, starting at 20 Hz) and the left-side vertical axis represents SPL in decibels (dB). Comparing FIG. 13 and FIG. 14, it is seen that the hearing system of FIG. 14 “fills in” a gap between the passive attenuation signal peak at 2 kHz and the high frequency reproduction signal plateau of about 7 kHz by summing the passive attenuation signal and the high frequency reproduction signal, thereby improving the linearity of the output signal. The improved linearity in turns allows for better control over the signal, so that raising and lowering the signal has a relatively uniform effect across a wider range of frequencies in FIG. 14 compared to FIG. 13.

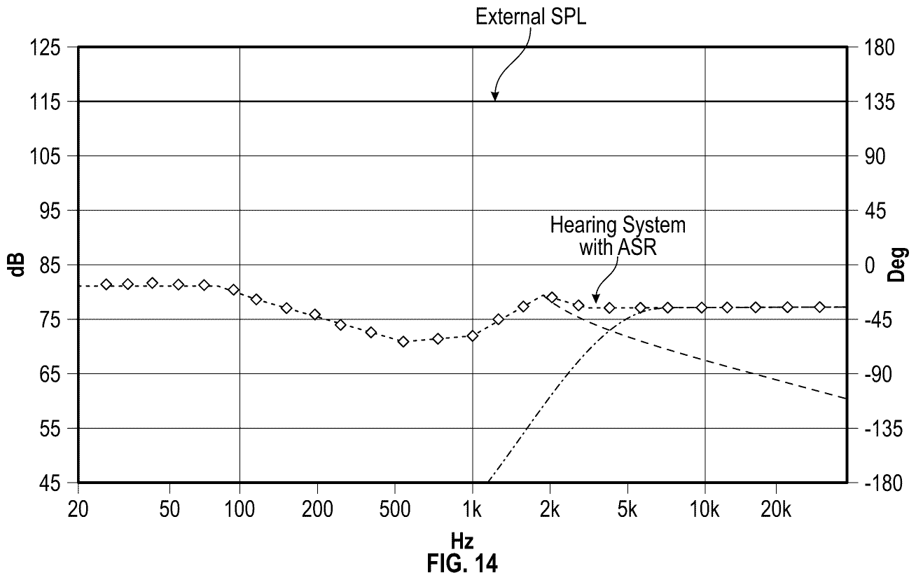

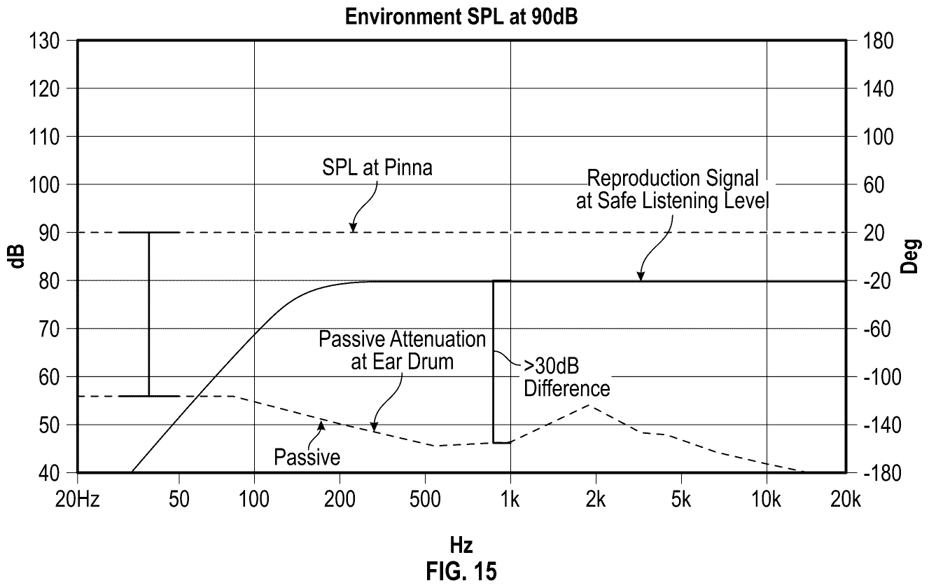

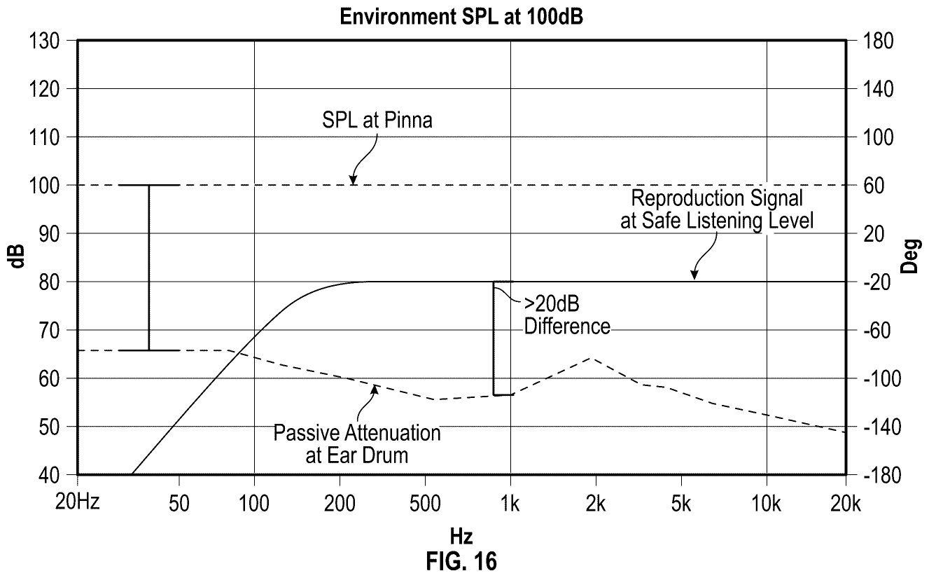

The wearer may want more environmental SPL due to passive attenuation attenuating more than desired in an office or other lower SPL setting. FIGS. 15-17 illustrate use cases with variable filter, compression and gain. The environmental SPL at the wearer's Cochlea 102 is 90 dB, 100 dB, and 120 dB, respectively, in FIGS. 15-17. In FIG. 15, the reproduced signal exceeds the passive attenuation alone at the ear drum at about 60 Hz, providing about a 30 dB increase at 1,000 Hz. In FIG. 16, the reproduced signal exceeds the passive attenuation alone at the ear drum at about 90 Hz, providing about a 20 dB difference at 1,000 Hz.

In FIG. 16, the environmental SPL is 100 dB, compared to the higher environmental SPL of 120 dB in FIG. 17. Passive attenuation in the two environments lowers the decibel level below 50 Hz by about 34 dB (i.e., 90 dB minus 56 dB in FIG. 15 and 100 dB minus 66 dB in FIG. 16), as shown by the “I” bars in the respective graphs. In both graphs, passive attenuation is non-linear. In FIG. 16, the passive attenuation signal throughout the entire range of frequencies is relatively low, never exceeding about 66 dB, such that the reproduction signal at safe listening level, which plateaus at about 80 dB, is greater than the passive attenuation signal throughout the frequencies of interest, e.g., 1 to 5 kHz. As a result, a full band compression operation may be considered more optimal for the particular 100-dB environment of FIG. 16. On the other hand, in FIG. 17 the passive attenuation signal is greater than the reproduction signal at safe listening level up to about 3.8 KHz.

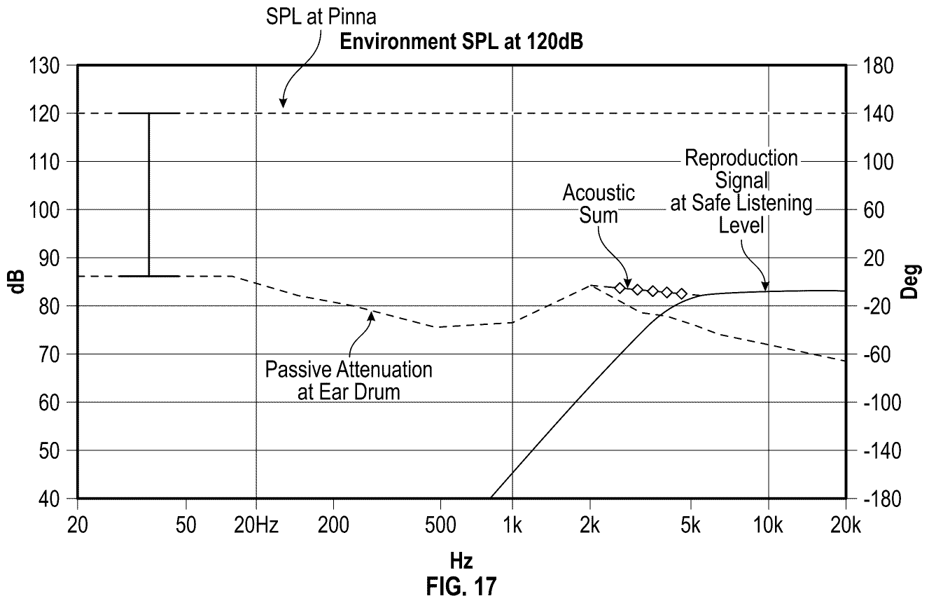

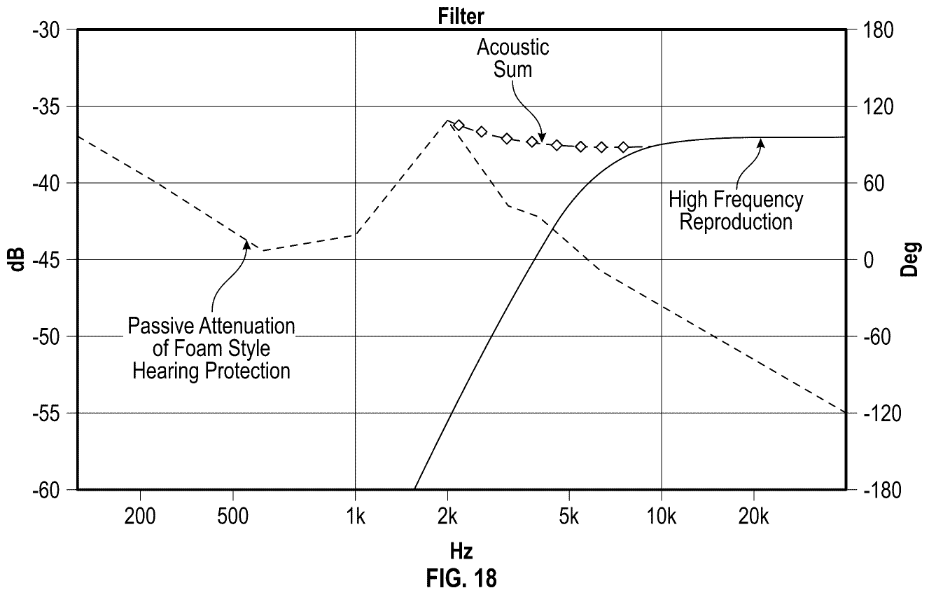

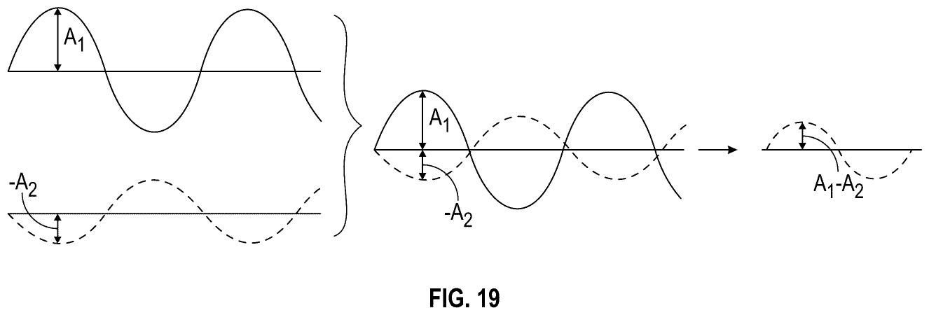

According to one or more embodiments, the ASR feature includes at least three main factors in combination. Referring now more particularly to FIG. 18, a passive attenuation of the structure blocking (or partially blocking) the ear canal is calculated. According to an embodiment, passive attenuation in the ear canal results partly from use of a foam-style hearing protection ear-piece component. The high frequencies of the sound detected by exterior microphone(s) on the outside facing portion of the earpiece are reproduced by the driver (or speaker) inside the canal with feedforward/feedback gain and compression signal processing. The low frequencies that leak through the passive attenuation structure can be attenuated with feedforward/feedback partial destructive interference where the leakage signal is summed with a lower amplitude cancellation signal generated by the dynamic transducer, as shown in FIG. 19.

According to an embodiment, the hearing protection system can automatically alternate between a hybrid active noise cancellation (ANC) mode and an ASR mode in real time depending on the listener's application needs, e.g., the environmental SPLs. For example, the user can utilize the device for telecommunications where hybrid ANC is used to cancel noise outside of the call signal. When the user ends the call, the device will automatically alternate to its ASR mode, for example, just in time for when the user walks past a train station or other high environmental SPL. An additional example may include a user listening to music on the device while they travel to a sporting event with the ANC prioritizing the music signal and cancelling extraneous noise. Once the user arrives at the sporting event and switches the music off, the ASR mode engages, reducing the SPL while preserving the signal integrity of the event. The alternating or switching between the ANC and the ASR modes may be automatically controlled by the DSP module or may be manually controlled, e.g., by the user.

According to an embodiment, active sound reduction is characterized by high-fidelity low-distortion compression, high-frequency environment reproduction (feedforward/feedback compression and gain); low-frequency attenuation (hybrid; feedforward and feedback), and conservation of hearing. In an embodiment, active noise cancellation is characterized by high-fidelity low-distortion music and media reproduction, clear and intelligible telecommunications, and hybrid operations (combination of feedforward and feedback).

According to an embodiment, the ASR system's contribution to hearing conservation is expanded by a continuous monitoring and parameter adjustment feature described as the Automated Safe Hearing Algorithm (ASHA). The embodied device monitors, logs, and stores the SPL dosage a user is exposed to over time. SPL metering often uses different weighting depending on amplitude and application. According to an embodiment, a weighting calculation incorporates the user's otoacoustically derived audiogram. For example, the A weighting curve may be modified based on its difference from a user's audiogram.

Once a pre-determined dosage limit has been exceeded, the device can (e.g., transparently) reduce the SPL at the ear canal, such as to an audiologist recommended limit or other value. This type of reduction is in addition to brief transient compression that may occur at any moment regardless of dosage level. The attack, decay, sustain, release, and threshold of brief transient compression may differ. An example of the basic conditions for the algorithm are detailed below.

SPL @ inner microphone<=80 dB SPL (A weighted), reproduction is equivalent to environment.

SPL @ inner microphone>80 dB SPL (A weighted), reproduction is 80 dB SPL (A weighted) @ ear canal.

SPL @ inner microphone=80 dB SPL (A weighted) for >8 hours, reproduction is <80 dB SPL (A weighted) @ ear canal.

In example embodiments, the above conditions can be further advanced by adjusting the desired SPL dosage and the desired amount of sound reduction to be engaged. Additional conditions can include presets such as a concert mode that provides a more musical tuning to its compression mechanics, a transportation mode, and a sporting event mode. With the use of multiple microphones on the outward facing portion of the device, beamforming may be used to prioritize a stage or voice.

In addition to the ASHA, the logged SPL data can be compared with a periodic logging of the user's otoacoustically derived audiogram to provide a continuous update on the state of the user's hearing. This data may be stored locally or through a cloud platform where the data can be provided to the user's audiologist.

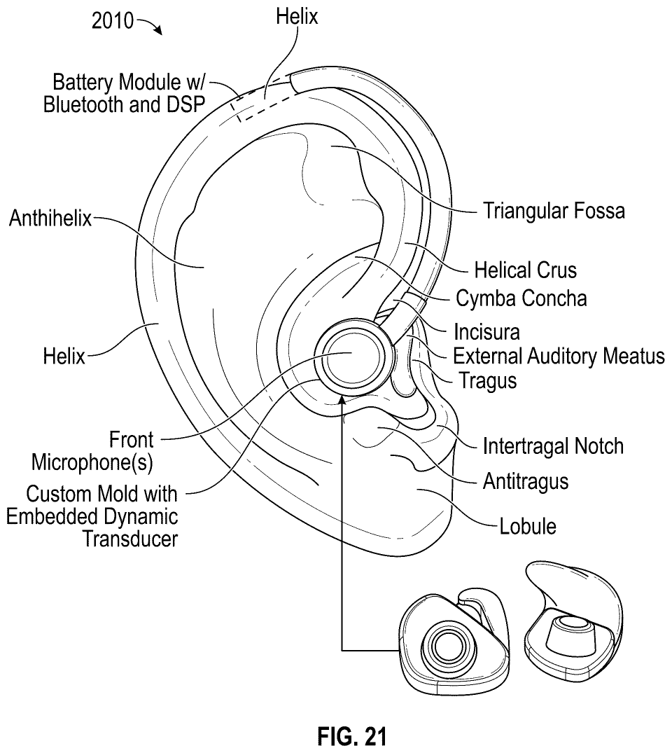

Referring now more particularly to FIGS. 20 and 21, embodiments of the wearable device 2010 and 2110 include: (i) at least one first (front or external) microphone (i.e., a first audio input transducer) configured to be outside an ear canal of a wearer; (ii) at least one second (internal) microphone (i.e., a second audio input transducer) configured to be inside the ear canal of the wearer; and (iii) a driver or speaker (i.e., an audio output transducer) configured to play (or produce) sound into the ear canal of the wearer. The hearing protection systems further include (iv) a controller (or DSP module) operatively connected to the first and second microphones and the driver. In an example embodiment, controller is configured to monitor exposure of the wearer to sound pressure level detected by at least the first (external) microphone, use the driver or speaker to reproduce high frequencies of the detected sound pressure level inside the ear canal of the ear of the wearer with feed forward/feedback gain and compression signal processing, determine passive attenuation of the hearing protection device by comparing the signals detected at the external and internal microphones, and/or attenuate low frequencies of the sound pressure level that leak through the passive attenuation using a feedforward/feedback partial destructive interference. In an embodiment, the controller 250 is incorporated into the worn device. In another embodiment, the controller is part of a separate device, such as a handheld device, that communicates (e.g., wirelessly or using one or more wires) with the wearable device. In another embodiment, the system comprises a fully functioning telecommunication device, e.g., capable of all of the functions of a contemporary smartphone.

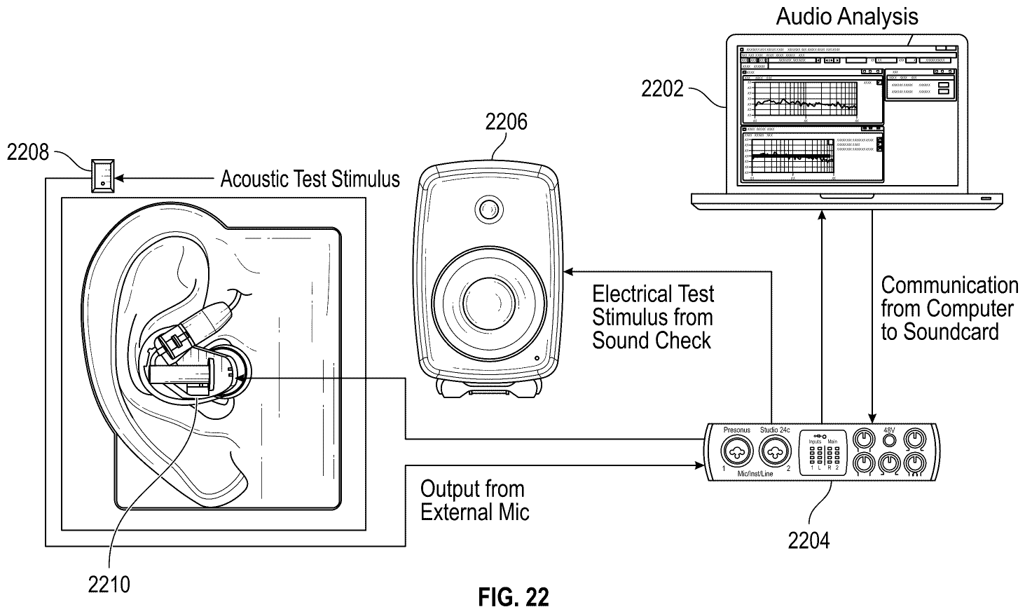

Referring now more particularly to FIG. 22, a testing system is generally designated by reference numeral 2200. The testing system 2200 includes a computer 2202, a soundcard 2204 that is in communication with the computer 202, an external speaker 2206 in communication with the sound card 2204 for generating an acoustic test stimulus 2208, and a wearable device 2210.

According to an embodiment, a device is provided as a physical in-ear tip piece. The device comprises microphones both outside of the ear canal and in the ear canal along with an audio output transducer (e.g., dynamic transducer such as a driver) that plays into the ear canal. This configuration of the device provides the capability to non-invasively monitor the listener's/wearer's exposure to sound pressure level and provides an automated system of protection that will be detailed herein.

Active Noise Cancellation (ANC) and Active Noise Reduction (ANR) are sometimes used interchangeably in the engineering and marketing of headphone devices since ANC, at most, can usually achieve around-40 dB of gain and thus does not achieve the perfect cancellation of noise. The systems, products, and methods according to example embodiments described herein clarify a distinction between ANC and Active Sound Reduction (ASR).

ASR is the use of an active hearing protection device that is intended to reduce the sound pressure level (SPL) of all sound incoming to the ear canal down to a selected or specified level at specific frequencies or the entire spectrum while preserving the original signals, such as to the highest degree possible, i.e., fidelity. In accordance with an example embodiment, an advantageous ASR system preserves the fidelity of all (or substantially all) signals such as music at a concert for a given user and is capable of automated parameter activation, optionally utilizing the individual user's/wearer's otoacoustically derived audiogram when selecting the SPL reduction parameters.

In an embodiment, the ASR feature would include three main factors in combination. The passive attenuation of the unit blocking the canal would be calculated. The high frequencies of the sound taken from microphones on the outside facing portion of the device would be reproduced by a dynamic transducer inside the canal with feedforward/feedback gain and compression signal processing. The low frequencies that leak through the passive attenuation can be attenuated with feedforward/feedback partial destructive interference where the leakage signal is summed with a lower amplitude cancellation signal generated by the dynamic transducer.

Applicant performed a study to investigate the feasibility of executing ASR by creating a development platform that included a digital audio workstation, an audio interface, a microphone, two different pairs of headphones, and an active loudspeaker, as shown in FIG. 22. Test subjects wore a Sennheiser circumaural closed HD280 headphone with an electret microphone placed near the outside of the ear. The loudspeaker played pink noise at 80 dBa, 76 dBa, and 70 dBa for 1-3 minutes for each level in an 18 ft. by 10 ft. room. The test subjects listened to the microphone signal routed into the headphone while a limiter in the digital audio workstation was tuned to reduce the signal being reproduced by 6 dB from the room SPL at the listening position. This test was repeated with a Shure SE215 in-ear headphone with foam tips.

It was found that HD280 circumaural headphones had poor passive attenuation. The conditioned microphone signal being reproduced was overwhelmed by the leakage of pink noise from the loudspeaker at all levels. Some of the leakage likely could be solved with low frequency partial destructive interference. The SE215 in-ear performed much better. Higher frequency reproduction of the signal combined with the passive attenuation of the in-ear resulted in what the subjects described as a “transparent” reduction in volume from 76 dBa in the room to approximately 70 dBa at the ear. This demonstrates that a basic in-ear design is capable of a combination of passive attenuation and high frequency reproduction (partial ASR) to result in a subjectively high-fidelity sound reduction in a moderate range of SPL.

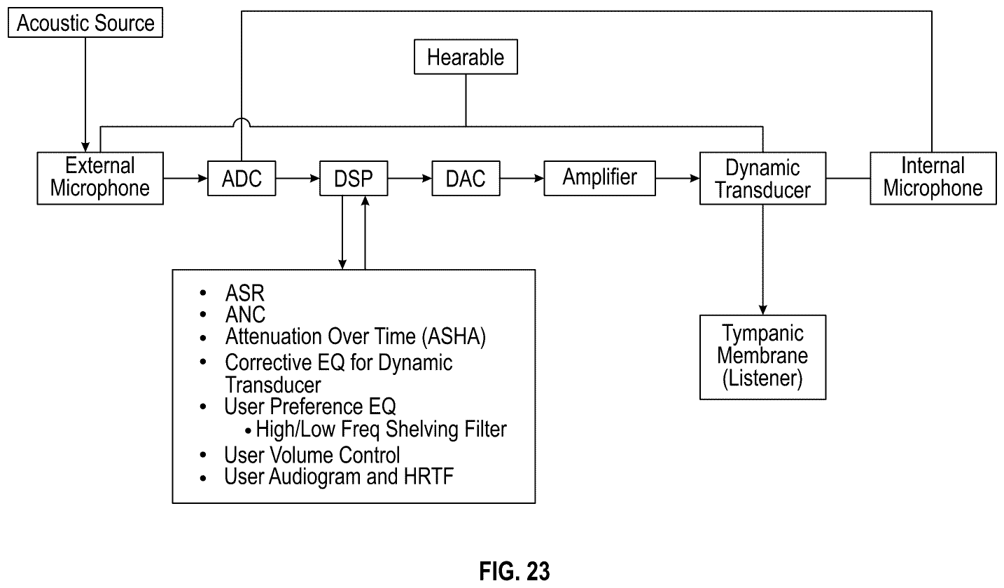

FIG. 23 illustrates a system, including hardware and general specifications, according to an example embodiment. The hardware and specifications of the embodiment include the following:
6 microphones or more, 2 outward facing, 1 inward facing for each ear (in some cases 8 microphones can be used, or any suitable number of microphones)
10 mm dynamic driver.
Sensitivity: 100 dB/mV (Presents cancellation limit for lower frequencies).
Frequencies response; 20 Hz-20 KHz
Total Harmonic Distortion (THD)<1%
Ergonomic sealable foam, silicone flange, or foam-silicone blended tips
Prescription molded tip and system available
Qualcomm aptX
Hybrid ANC
ASR with ASHA
Adaptive Acoustic Technology
Concert, Sport, Transportation, & Passthrough modes

FIG. 24 illustrates an example embodiment of the signal processing method including, specifically, how signals are selected for playback and which signals are selected for playback or reproduction for the user. As noted above, the hearing protection system (200, 300) can be used to enjoy standard forms of audio programming or music playback since in the example embodiments illustrated in FIG. 3-8 the digital input audio signal can be from external microphone 308, or it can be a playback input signal 401 (e.g., from a wireless receiver 400 and a media source 403), which are introduced into DSP module 316 directly. As shown in FIG. 24, if the hearing protection system is operated in a hearing protection mode, DSP 316 is programmed to perform the condition detection and action steps described in the “External SPL” section. And when the hearing protection system is operated in Phone media playback mode, the DSP is programmed to perform the steps described in the “Phone media” section, which does still include hearing protection steps, including logging SPL at the user's ear drum.

It should be understood that the hardware, software, and system requirements are not limited to the example embodiments described above.

Applications for the devices, systems, and methods described herein include everyday use, concerts, urban transportation, occupational hazard, potential treatment for medically diagnosed sensitivities (e.g., hyperacusis, autistic sensory overstimulation, etc.). The aforementioned listed uses are by way of example and not exhaustive of possible uses and environments suitable for embodiments disclosed herein.

In the context of software, the operations described herein represent non-transitory program code (e.g., computer-executable instructions, datastores, etc.), which may be stored on one or more tangible, computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the process.

The term “module” refers broadly to locally executed software, software executed in the cloud, hardware, or firmware components (or any combination thereof). Modules are typically functional components that can generate useful data or other output using specified input(s). A module may or may not be self-contained. An application program (also called an “application”) may include one or more modules, or a module may include one or more applications. Generally, modules may include programs, components, objects, routines, logic, data structures, and so on that perform and implement particular tasks. A task may be practiced in distributed cloud computing environments, where tasks are performed by remote processing devices that are linked through a communications network. Modules may be located in a local computer system storage medium/media and/or remote computer system storage medium/media. Modules generally carry out the functions and/or methodologies of embodiments.

Modules may be stored in, for example, memory, operating systems, one or more application programs, other program modules, and program data.

The memory may include computer-readable storage media (“CRSM”), which may be any available physical media accessible by the processor to execute instructions stored on the memory. In an implementation, CRSM may include random access memory (“RAM”), flash memory and/or cache memory. In other implementations, CRSM may include, but is not limited to, read-only memory (“ROM”), electrically erasable programmable read-only memory (“EEPROM”), or any other medium which can be used to store the desired information and which can be accessed by the processor.

Computer programs are stored in memory, which may be physically present on the hearing protection device or an external device/system in communication with the hearing protection device. Computer programs may also be received via a communication interface. Such computer programs, when run, enable the computer system to perform the features of the present embodiments as discussed herein. In particular, the computer programs, when run, enable the processing unit to perform the features of the computer system. Accordingly, such computer programs represent controllers of the computer system. For example, a storage system may be provided for reading from and writing to a non-removable, non-volatile magnetic media (e.g., a hard drive). A magnetic disk drive for reading from and writing to a removable, non-volatile magnetic disk (e.g., a disk), and an optical disk drive for reading from or writing to a removable, non-volatile optical disk such as a CD-ROM, DVD-ROM or other optical media can be provided. In such instances, each can be connected to bus by one or more data media interfaces.

Terms “computer program medium,” “computer usable medium,” and “computer readable medium” are used to generally refer to media such as main memory, including RAM, cache, and storage system, such as a removable storage drive and a hard disk installed in a hard disk drive. Computer readable storage medium can be a tangible device that can retain and store instructions for use by an instruction execution device. The computer readable storage medium may be, for example and without limitation, an electronic storage device, a magnetic storage device, an optical storage device, an electromagnetic storage device, a semiconductor storage device, or any suitable combination of the foregoing. A computer readable storage medium, as used herein, is to be construed as non-transitory, and is not to be construed as being transitory signals per se.

While particular embodiments have been shown and described, it will be understood to those skilled in the art that based upon the teachings herein, changes and modifications may be made without departing from its broader aspects. Therefore, the appended pseudo claim statements are to encompass within their scope all such changes and modifications as are within the true spirit and scope of the embodiments. Furthermore, it is to be understood that the embodiments are solely defined by the appended pseudo claim statements. It will be understood by those with skill in the art that if a specific number of an introduced pseudo claim statement element is intended, such intent will be explicitly recited in the pseudo claim statements, and in the absence of such recitation no such limitation is present. For non-limiting example, as an aid to understanding, the following appended pseudo claim statements contain usage of the introductory phrases “at least one” and “one or more” to introduce pseudo-claim statement elements. However, the use of such phrases should not be construed to imply that the introduction of a pseudo claim statement element by the indefinite articles “a” or “an” limits any particular pseudo claim statement containing such introduced pseudo claim statement element to the embodiments containing only one such element, even when the same pseudo claim statement includes the introductory phrases “one or more” or “at least one” and indefinite articles such as “a” or “an”; the same holds true for the use in the pseudo claim statements of definite articles. As used herein, the term “and/or” means either or both (or any combination or all of the terms or expressed referred to).

Embodiments described herein may be, among other things, a system, a computer program product, and a method. Selected aspects and features of example embodiments described herein may take the form of an entirely hardware embodiment, an entirely software embodiment or an embodiment combining software and/or hardware aspects that may all generally be referred to herein as a “circuit,” “module” or “system.” Furthermore, aspects of example embodiments may take the form of computer program product embodied in a computer readable storage medium (or media) having computer readable program instructions thereon for causing a processor to carry out aspects of the present embodiments.

Aspects are described herein with reference to flowchart illustrations and/or block diagrams of systems, computer program products, and methods according to embodiments. Each block of the flowchart illustrations, block diagrams, and combinations of blocks can be implemented by computer readable program instructions. The computer readable program instructions may be provided to a processor to produce a machine, such that the instructions, which are executed via the processor, implement the functions/acts specified in the flowcharts and/or block diagram block or blocks. These computer readable program instructions may also be stored in memory, such as described herein, such that the computer readable storage medium having instructions stored therein comprises an article of manufacture including instructions which implement aspects of the function/act specified in the flowchart and/or block diagram block or blocks.

The flowcharts and block diagrams in the Figures illustrate examples of the architecture, functionality, and operation of certain implementations of systems, computer program products, and methods in accordance with certain embodiments. In this regard, each block in the flowchart or block diagrams may represent a module, segment, or portion of instructions, which comprises one or more executable instructions for implementing the specified logical function(s). In some alternative implementations, the functions noted in the block may occur in a different order or sequence than that noted in the figures. Further, two or more blocks shown in succession may, in fact, be executed substantially concurrently, or the blocks may sometimes be executed in the reverse order. In other implements, one or more blocks may be omitted, and/or additional blocks not shown in the accompanying Figures may be included within the scope of the systems, computer program products, methods, and other embodiments described herein.

The corresponding structures, materials, acts, and equivalents of all means or step plus function elements in the pseudo claim statements below are intended to include any structure, material, or act for performing the function in combination with other pseudo claim statement elements as specifically claimed. The description of the present embodiments has been presented for purposes of illustration and description, but is not intended to be exhaustive or limited to the embodiments in the form disclosed. Many modifications and variations will be apparent to those of ordinary skill in the art without departing from the scope and spirit of the embodiments. The embodiments were chosen and described in order to best explain the principles of the embodiments and the practical application, and to enable others of ordinary skill in the art to understand the embodiments for various embodiments with various modifications and combinations with one another as are suited to the particular use contemplated. Accordingly, the scope of protection of the embodiment(s) is limited only by the following pseudo claim statements and their equivalents.

Having described example embodiments of a new and improved apparatus and method, it is believed that other modifications, variations and changes will be suggested to those skilled in the art in view of the teachings set forth herein. It is therefore to be understood that all such variations, modifications and changes are believed to fall within the scope of the disclosure.

### EXAMPLE EMBODIMENTS

Embodiment 1. An active sound-reduction, hearing protection system (200 or 300) comprising:
an earpiece (e.g., 202 or 302) wearable in an ear canal of a subject, comprising:
   a passive attenuation structure (204, 304) configured to sit at least partially within the ear canal (104, 106) when the earpiece is worn;
   a first input transducer (308) configured to be acoustically coupled to environmental surroundings of the subject when the earpiece is worn to receive environmental sounds (302) and generate a first input audio signal (314) corresponding to the environmental sounds;
   an output transducer (330) configured to be acoustically coupled to an inner volume of the ear canal when the earpiece is worn to receive an output audio signal (324) and generate playback output sounds (332) based on the output audio signal (324), the playback output sounds (332) emitted into the ear canal when the earpiece is worn; and
   a second input transducer (338) configured to be acoustically coupled to the inner volume of the ear canal when the earpiece is worn to receive an acoustic sum (334) comprising the playback output sounds (332) and leakage sounds (306) and generate a second input audio signal (340), the leakage sounds originating from the environmental surroundings yet not acoustically blocked from the inner volume of the ear canal by the earpiece;
memory; and
a digital signal processing (DSP) module (316) operatively coupled to the memory, the DSP module (316) configured to:
   apply short-term sound reduction (404) to the first input audio signal (314) and/or the second input audio signal (340) in response to transient high-level sound pressure levels;
   using the second input audio signal, accumulate an exposure history of the ear canal to the acoustic sum (334) over a period of time, and record the exposure history in the memory; and
   apply long-term sound reduction (408) to the first input audio signal (314) and/or the second input audio signal (340) in response to non-transient high-level sound pressure levels based upon the exposure history, and
   generate the output audio signal (324) based on the application of the short-term sound reduction and/or the long-term sound reduction to the first input audio signal (314) and/or the second input audio signal (340).

Embodiment 2. The active sound-reduction, hearing protection system of Embodiment 1, wherein:
the short-term sound reduction comprises short-term compression (404); and
the long-term sound reduction comprising long-term compression (408) and/or attenuation (414).

Embodiment 3. The active sound-reduction, hearing protection system of Embodiment 1, wherein the period of time is within a range of 1 hour to 24 hours.

Embodiment 4. The active sound-reduction, hearing protection system of Embodiment 1, wherein the period of time is greater than 24 hours.

Embodiment 5. The active sound-reduction, hearing protection system of Embodiment 1, wherein the first and second input transducers (e.g., 308 and 336) share a common analog-to-digital converter (e.g., 326).

Embodiment 6. The active sound-reduction, hearing protection system of Embodiment 1, wherein the short-term sound reduction and the long-term sound reduction comprise single-band short-term compression and single-band long-term compression.

Embodiment 7. The active sound-reduction, hearing protection system of Embodiment 1, wherein the short-term sound reduction and the long-term sound reduction comprise short-term multi-band compression and long-term multi-band attenuation, respectively.

Embodiment 8. The active sound-reduction, hearing protection system of Embodiment 1, wherein the long-term attenuation comprises attenuation via partial destructive interference of low frequencies of the second input audio signal corresponding to the leakage sounds.

Embodiment 9. The active sound-reduction, hearing protection system of Embodiment 1, wherein the DSP module is further configured to, using the second input audio signal, reproduce high frequencies of the environmental sounds that are lost to passive attenuation.

Embodiment 10. A computer-implemented hearing protection method, comprising:
(a) providing a first (e.g. left) earpiece wearable in a first ear canal of a subject, said first earpiece comprising: a first passive attenuation structure (304) configured to sit at least partially within the first ear canal when the earpiece is worn;
a first input transducer (308) configured to be acoustically coupled to environmental surroundings of the subject when the first earpiece is worn to receive environmental sounds (302) and generate a first input audio signal (314) corresponding to the environmental sounds;
a first output transducer (330) configured to be acoustically coupled to an inner volume of the ear canal when the first earpiece is worn to receive a first output audio signal (324) and generate first playback output sounds (332) based on the first output audio signal (324), the first playback output sounds (332) emitted into the first ear canal when the earpiece is worn; and
a second input transducer (338) configured to be acoustically coupled to the inner volume of the first ear canal when the earpiece is worn to receive a first acoustic sum (334) comprising the first playback output sounds (332) and first leakage sounds (306) and generate a second input audio signal (340), the first leakage sounds originating from the environmental surroundings yet not acoustically blocked from the inner volume of the first ear canal by the earpiece;
said first earpiece further comprising circuits responsive to a microprocessor or computer and a digital signal processing module and system memory configured and programmed to do one or more of the following:
(i) Sense a leakage level using the internal transducer or microphone 336;
(ii) Determine and set a high pass Fc and long-term compressor threshold or output gain attenuation for the user;
(iii) Feed a first external transducer signal through, for example, a 2 stage compressor (full or multiband);
(iv) Play back the compressed signal through the first driver transducer 330 into the user's first ear canal;
(v) Determine or Calculate: Driver Output+Ear Plug Leakage=Total SPL (where total SPL in the user's first ear canal is acoustically Summed at the internal transducer microphone 336 and also at the user's ear drum);
(vi) Continually monitor said first (e.g., left ear) Total SPL by internal transducer or microphone 336; and
(vii) If user-specific safe level is exceeded, Reduce long term threshold for subject user, where, in the case of multiband compression, for example the internal mic signal does not feed the long-term compressor threshold (of step (ii)), but instead feeds the output gain attenuation for total SPL;
(b) recording said subject user's first ear canal's Total SPL exposure data as updated information for said user in said system memory.

Embodiment 11. The computer-implemented hearing protection method of Embodiment 10, comprising one or more of:
(c) in response to said user's Total SPL exposure data, automatically determining updated user-specific or personalized optimal sound characteristics for said user to cater to the user's then-extant unique hearing; and
(d) in response to said user's Total SPL exposure data, automatically applying said user-specific or personalized optimal sound characteristics for said user to said digital signal processing module to change the signal to said first transducer to more closely approximate said user-specific or personalized optimal sound characteristics for said user; and
(e) providing a second (e.g. right) earpiece wearable in a second ear canal of a subject, said second earpiece comprising elements similar to the first earpiece;
(f) recording said subject user's second ear canal's Total SPL exposure data as updated information for said user in said system memory; and
(g) in response to said user's right Total SPL exposure data, automatically determining updated user-specific or personalized optimal sound characteristics for said user to cater to the user's then-extant unique hearing; and
(d) in response to said user's right side Total SPL exposure data, automatically applying said user-specific or personalized optimal sound characteristics for said user to said digital signal processing module to change the signal to said first right transducer to more closely approximate said user-specific or personalized optimal sound characteristics for said user.

Embodiment 12. An active sound-reduction, hearing protection system (200, 300) comprising:
an earpiece wearable in an ear canal of a subject, comprising:
   a passive attenuation structure (304) configured to sit at least partially within the ear canal when the earpiece is worn;
   a first input transducer (308) configured to be acoustically coupled to environmental surroundings of the subject when the earpiece is worn to receive environmental sounds (302) and generate a first input audio signal (314) corresponding to the environmental sounds;
   an output transducer (330) configured to be acoustically coupled to an inner volume of the ear canal when the earpiece is worn to receive an output audio signal (324) and generate playback output sounds (332) based on the output audio signal (324), the playback output sounds (332) emitted into the ear canal when the earpiece is worn; and
   a second input transducer (338) configured to be acoustically coupled to the inner volume of the ear canal when the earpiece is worn to receive an acoustic sum (334) comprising the playback output sounds (332) and leakage sounds (306) and generate a second input audio signal (340), the leakage sounds originating from the environmental surroundings yet not acoustically blocked from the inner volume of the ear canal by the earpiece;
memory; and
a digital signal processing (DSP) module (316) operatively coupled to the memory, the DSP module (316) configured to:
   use the second input audio signal, accumulate an exposure history of the ear canal to the acoustic sum (334) over a period of time; and
   apply long-term sound reduction (408) to the first input audio signal (314) and/or the second input audio signal (340) in response to non-transient high-level sound pressure levels based upon the exposure history, and
   generate the output audio signal (324) based on the application of, at least, the long-term sound reduction to the first input audio signal (314) and/or the second input audio signal (340).

The following is claimed:

1. A hearing protection system comprising:
an earpiece wearable in an ear canal of a subject, the earpiece comprising:
a passive attenuation structure;
an external microphone positioned to receive environmental sounds when the earpiece is worn and configured to generate a first input audio signal corresponding to the environmental sounds;
an output transducer positioned and configured to deliver playback output sounds into the ear based on an output audio signal when the earpiece is worn; and
an internal microphone positioned to receive an acoustic sum comprising the playback output sounds and leakage sounds that include environmental sounds not acoustically blocked from the ear canal by passive attenuation structure of the earpiece, and configured to generate a second input audio signal corresponding to the acoustic sum;
memory; and
a digital signal processing (DSP) module operatively coupled to the memory, the DSP module configured to:
use the second input audio signal to accumulate an exposure history of the ear to the acoustic sum over a period of time, and record the exposure history in the memory;
apply long-term sound reduction to the first input audio signal and/or the second input audio signal in response to sound pressure levels based at least in part on the exposure history; and
generate the output audio signal based on the application of at least the long-term sound reduction to the first input audio signal and/or the second input audio signal.

2. The hearing protection system of claim 1, wherein the DSP module is configured to apply short-term sound reduction to the first input audio signal and/or the second input audio signal in response to transient sound pressure levels above a threshold.

3. The hearing protection system of claim 2, wherein the short-term sound reduction comprises short-term compression, and the long-term sound reduction comprising long-term compression and/or attenuation.

4. The hearing protection system of claim 2, wherein the short-term sound reduction comprises single-band short-term compression, and the long-term sound reduction comprises single-band long-term compression.

5. The hearing protection system of claim 2, wherein the short-term sound reduction comprises short-term multi-band compression, and the long-term sound reduction comprises long-term multi-band attenuation.

6. The hearing protection system of claim 1, wherein the long-term sound reduction comprises single-band long-term compression.

7. The hearing protection system of claim 1, wherein the long-term sound reduction comprises long-term multi-band attenuation.

8. The hearing protection system of claim 1, wherein the long-term sound reduction comprises attenuation via partial destructive interference of frequencies of the second input audio signal corresponding to the leakage sounds.

9. The hearing protection system of claim 8, wherein the frequencies are below a threshold.

10. The hearing protection system of claim 1, wherein the DSP module is configured to, using the second input audio signal, reproduce frequencies of the environmental sounds that are lost to passive attenuation.

11. The hearing protection system of claim 10, wherein the frequencies are above a threshold.

12. The hearing protection system of claim 1, wherein the period of time is within a range of 1 hour to 24 hours.

13. The hearing protection system of claim 1, wherein the period of time is greater than 24 hours.

14. The hearing protection system of claim 1, wherein the external microphone and the internal microphone share a common analog-to-digital converter.

15. The hearing protection system of claim 1, wherein the DSP module is configured to sense a leakage level using at least the internal microphone.

16. The hearing protection system of claim 1, wherein the DSP module is configured to apply more compression to a lower range of frequencies and to apply less or no compression to a higher range of frequencies.

17. The hearing protection system of claim 1, wherein the DSP module is configured to determine a direction of a sound and to determine an amount of sound reduction to the sound based at least in part on the determined direction of the sound.

18. The hearing protection system of claim 1, configured to switch between an active noise cancelation mode and an active sound reduction mode.

19. A hearing protection method comprising:
providing a first earpiece wearable in a first ear of a subject, said first earpiece comprising:
a passive attenuation structure;
an external microphone positioned to receive environmental sounds when the earpiece is worn and configured to generate a first input audio signal corresponding to the environmental sounds;
an output transducer positioned and configured to deliver playback output sounds into the ear based on an output audio signal when the earpiece is worn; and
an internal microphone positioned to receive an acoustic sum comprising the playback output sounds and leakage sounds that include environmental sounds not acoustically blocked from the ear canal by passive attenuation structure of the earpiece, and configured to generate a second input audio signal corresponding to the acoustic sum; and
using a digital signal processing (DSP) module and memory to:
use the second input audio signal to accumulate an exposure history of the ear to the acoustic sum over a period of time, and record the exposure history in the memory;
apply long-term sound reduction to the first input audio signal and/or the second input audio signal in response to sound pressure levels based at least in part on the exposure history; and
generate the output audio signal based on the application of at least the long-term sound reduction to the first input audio signal and/or the second input audio signal.

20. The hearing protection method of claim 19, comprising using the DSP module to apply short-term sound reduction to the first input audio signal and/or the second input audio signal in response to transient sound pressure levels above a threshold.
